(19) United States
(12) Patent Application Publication
(10) Pub. No.: US 2007/0292404 A1
(43) Pub. Date: Dec. 20, 2007

(54) Antimicrobial polymer conjugates

(75) Inventors: Scott M. Walsh, Poughquag, NY (US); Anjali G. Shah, North Potomac, MD (US); James J. Mond, Silver Spring, MD (US); Andrew Lees, Silver Spring, MD (US); Joseph J. Drabick, Palmyra, PA (US)

Correspondence Address: MEDLEN & CARROLL, LLP, 101 HOWARD STREET, SUITE 350, SAN FRANCISCO, CA, 94105, US

(73) Assignee: Biosynexus Incorporated, Gaithersburg, MD (US)

(21) Appl. No.: 11/728,911

(22) Filed: Mar. 27, 2007

Related U.S. Application Data
(60) Provisional application No. 60/786,188, filed on Mar. 27, 2006.

(51) Int. Cl. A61K 38/48; A61P 31/04

(52) U.S. Cl. 424/94.3

(57) ABSTRACT

The present invention relates to the conjugation of antimicrobial agents to water-soluble polymers to improve their clinical properties in terms of their pharmacokinetics, pharmacodynamics, and reduced immunogenicity. More specifically, the present invention relates to the conjugation of antimicrobial agents such as lysostaphin to poly(alkylene oxides), such as poly(ethylene glycol) (PEG).

The present invention claims priority to U.S. Provisional Patent Application Ser. No. 60/786,188 filed Mar. 27, 2006, hereby incorporated by reference in its entirety.

### FIELD OF THE INVENTION

The present invention relates to the conjugation of antimicrobial agents to water-soluble polymers to improve their clinical properties in terms of their pharmacokinetics, pharmacodynamics, and reduced immunogenicity. More specifically, the present invention relates to the conjugation of antimicrobial agents such as lysostaphin to poly(alkylene oxides) (e.g., polyethylene glycol (PEG)).

### BACKGROUND OF THE INVENTION

Lysostaphin is a potent antimicrobial agent first identified in Staphylococcus simulans (formerly known as S. staphylolyticus). Lysostaphin is a bacterial glycal-glycine endopeptidase capable of cleaving the cross-linking polyglycine cross-bridges in the cell walls of bacteria (e.g., Staphylococci), and is therefore highly lethal thereto. Expressed in a single polypeptide chain, lysostaphin has a molecular weight of approximately 27 kDa.

The cell wall bridges of Staphylococcus aureus contain high levels of glycine (e.g., cross-linked polyglycine bridges), and thus lysostaphin is particularly effective in lysing S. aureus. Lysostaphin is also able to lyse other staphylococci including Staphylococcus epidermidis.

S. aureus is a highly virulent human pathogen. It is the cause of a variety of human diseases, ranging from localized skin infections to life-threatening bacteremia and infections of vital organs. If not rapidly controlled, a S. aureus infection can spread quickly from the initial site of infection to other organs. Although the foci of infection may not be obvious, organs particularly susceptible to infection include the heart valves, kidneys, lungs, bones, meninges and the skin (e.g., in burn patients).

Staphylococcal infections, such as those caused by S. aureus, are a significant cause of morbidity and mortality, particularly in settings such as hospitals, schools, and infirmaries. Patients particularly at risk include infants, the elderly, the immunocompromised, the immunosuppressed, and those with chronic conditions requiring frequent hospital stays.

Patients also at risk of acquiring staphylococcal infections include those undergoing inpatient or outpatient surgery, those within an Intensive Case Unit (ICU), on continuous hemodialysis, with HIV infection, with AIDS, burn victims, people with diminished immunity (e.g., resulting from drug treatment or disease), the chronically ill or debilitated patients, geriatric subjects, infants with immature immune systems, and people with intravascular (e.g., implanted) devices.

Small proteins (e.g., less than about 70 kDa), such as lysostaphin, may have a relatively short half-life in blood after intravenous injection. Lysostaphin's rapid clearance from circulation may reduce its efficacy. At the same time, because it is derived from a bacterial species and therefore foreign to any mammalian species, lysostaphin may also have undesired immunogenicity, which could further stimulate its clearance from the blood stream, especially in subjects that have had previous exposure to lysostaphin. Thus, lysostaphin's short circulating half-life cannot be effectively countered by increasing the amount or frequency of dosage. Further, there may be deleterious effects associated with administration of lysostaphin to an individual with anti-lysostaphin antibodies. There exists a need for improved means by which the circulating half-life of lysostaphin may be increased without increasing the amount or frequency of administration, and/or a means to potentially reduced the immunogenicity of lysostaphin when administered as a therapy or prophylaxis for staphylococcal infections or colonization. For example, it would be desirable to increase the circulating half-life of lysostaphin as well as reducing its capacity to induce an antibody response while concurrently not significantly altering lysostaphin's antimicrobial activity.

### SUMMARY OF THE INVENTION

The present invention relates to the conjugation of antimicrobial agents to water-soluble polymers to improve their clinical properties in terms of their pharmacokinetics, pharmacodynamics, and reduced immunogenicity. In some embodiments, the present invention relates to the conjugation of lysostaphin to poly(alkylene oxides) (e.g., polyethylene glycol (PEG)).

In some embodiments, the present invention provides for polymer conjugation of lysostaphin to increase circulating half-life in vivo while retaining antimicrobial activity. Lysostaphin so modified may thus be used to treat or prevent infection (e.g., at a reduced and/or less frequent dosage than an unmodified lysostaphin).

In addition to increasing circulating half-life while retaining antimicrobial activity, other advantages obtained by polymer conjugation include, but are not limited to, decreased antibody binding, increased efficacy (e.g., for killing or prohibiting growth of bacteria), decreased immunogenicity, increased tissue penetration and reduced binding to circulatory system surfaces.

In some embodiments, the present invention provides water-soluble polymers (e.g., polyethylene glycol (PEG)) conjugated to lysostaphin, such that at least a portion of the antimicrobial activity of lysostaphin is retained. In some embodiments, at least 50%, at least 60%, at least 70%, at least 75%, at least 80%, at least 85%, at least 90%, at least 95%, at least 97% or more of lysostaphin activity is retained post conjugation to the water-soluble polymer compared to un-conjugated lysostaphin. In some embodiments, lysostaphin may retain more (e.g., greater than 97%) or less (e.g., less than 50%) antimicrobial activity after conjugation to a water-soluble polymer. The present invention is not limited by the route or type of administration of a lysostaphin conjugate of the present invention. Indeed, a variety of routes of administration are contemplated to be useful including, but not limited to, ophthalmic, oral, transdermal and/or topical, nasal, into the lungs (e.g., via an inhalant), mucosal (e.g., vaginal or nasal mucosa, rectal, via the ear, by injection (e.g., intravenously, subcutaneously, intratumorally, intraperitoneally, etc.) and the like. In some embodiments, one or more other antimicrobial agents are co-administered with a lysostaphin conjugate (e.g., PEG-lysostaphin conjugate) of the present invention. The present invention is not limited to the type of antimicrobial agent co-administered. Indeed, a variety of antimicrobial agents are contemplated to be useful for co-administration with a composition of the present invention including, but not limited to, chemicals, peptides, proteins and lipopeptides (e.g., that may, upon contacting a microbe in a host, kill the microbe by any of a variety of techniques or inhibit microbial metabolism, without damaging host cells or tissues or eliciting a harmful host response).

The present invention is not limited by the type of water-soluble polymer to which lysostaphin is conjugated. For example, water-soluble polymers include, but are not limited to, poly(alkylene oxides), polyoxyethylated polyols and poly(vinyl alcohols). Poly(alkylene oxides) include, but are not limited to, PEGs, poloxamers and poloxamines. The present invention is not limited by the type of conjugation utilized (e.g., to connect a lysostaphin molecule to one or more water-soluble polymers (e.g. PEG)). In some embodiments, a poly(alkylene oxide) is conjugated to a free amino group via an amide linkage (e.g., formed from an active ester (e.g., the N-hydroxysuccinimide ester)) of the poly(alkylene oxide). In some embodiments, an ester linkage remains in the conjugate after conjugation. In some embodiments, linkage occurs through a lysine residue present in the lysostaphin molecule. In some embodiments, conjugation occurs through a short-acting, degradable linkage. The present invention is not limited by the type of degradable linkage utilized. Indeed, a variety of linkages are contemplated to be useful in the present invention including, but not limited to, physiologically cleavable linkages including ester, carbonate ester, carbamate, sulfate, phosphate, acyloxyalkyl ether, acetyl, and ketal linkages. In some embodiments, lysostaphin is conjugated to PEG utilizing any of the methods, reagents and/or linkages described in U.S. Pat. Nos. 4,424,311; 5,672,662; 6,515,100; 6,664,331; 6,737,505; 6,894,025; 6,864,350; 6,864,327; 6,610,281; 6,541,543; 6,515,100; 6,448,369; 6,437,025; 6,432,397; 6,362,276; 6,362,254; 6,348,558; 6,214,966; 5,990,237; 5,932,462; 5,900,461; 5,739,208; 5,446,090 and 6,828,401; and WO 02/02630 and WO 03/031581, each of which is herein incorporated by reference in its entirety. In some embodiments, a conjugate of the present invention is produced by a third party (e.g., NEKTAR, San Carlos, Calif.). In some embodiments, the conjugate comprises a cleavable linkage present in the linkage between the polymer and lysostaphin (e.g., such that when cleaved, no portion of the polymer or linkage remains on the lysostaphin molecule). In some embodiments, the conjugate comprises a cleavable linkage present in the polymer itself (e.g., such that when cleaved, a small portion of the polymer remains on the lysostaphin molecule). In some embodiments, the PEG-lysostaphin conjugate is purified after conjugation. The present invention is not limited by the type of purification process utilized. Indeed, a variety of processes may be utilized including, but not limited to, ion exchange chromatography, hydrophobic charge induction chromatography, size exclusion chromatography, and other methods well known in the art. The present invention is not limited by the type of PEG molecule utilized. Indeed, a variety of PEG molecules are contemplated to be useful for conjugation to a lysostphin molecule of the present invention including, but not limited to, linear or straight chained PEG or branched PEG and may have a molecular weight of between about 10 and about 500 kDa (e.g., in some embodiments, is between 20-100 kDa), although a PEG molecule conjugated to a lysostaphin molecule may be larger (e.g., greater than 500 kDa) or smaller (e.g., less than 10 kDa).

The present invention also provides a method for the prophylactic or therapeutic treatment of a microbial infection or colonization in a subject (e.g., a mammal) by administering to the subject an effective amount of a pharmaceutical preparation comprising an antimicrobial conjugate (e,g, PEG-lysostaphin conjugate) of the present invention (e.g., comprising a pharmaceutically acceptable carrier). The present invention is not limited by the type of bacteria treated. Indeed, a variety of bacteria are contemplated to be treatable (e.g., killed or growth inhibited) by a antimicrobial conjugate of the present invention including bacterial species with a peptidoglycan layer containing polyglycine bridges (e.g., that are enzymatically cleaved by a PEG-lysostaphin conjugate). In some embodiments, the bacteria are of the genus Staphylococcus (e.g., S. aureus).

In some embodiments, the present invention provides a composition comprising polyethylene glycol (PEG) conjugated to lysostaphin or a lysostaphin analogue, wherein the conjugate comprises a degradable linkage (e.g., an ester linkage), wherein at least a portion of the antimicrobial activity of the lysostaphin or lysostaphin analogue is retained. In some embodiments, the lysostaphin or lysostaphin analogue conjugated to polyethylene glycol through a degradable linkage has a longer in-vivo half-life than non-conjugated lysostaphin or lysostaphin analogue. In some embodiments, the lysostaphin or lysostaphin analogue is capable of cleaving cross-linked polyglycine bridges in the cell wall peptidoglycan of staphylococci. In some embodiments, conjugating the lysostaphin or lysostaphin analogue to the polyethylene glycol permits a greater serum concentration of lysostaphin or lysostaphin analogue than is achievable for non-conjugated lysostaphin or lysostaphin analogue. In some embodiments, the lysostaphin or lysostaphin analogue is a recombinant lysostaphin or lysostaphin analogue. In some embodiments, the lysostaphin is naturally derived. In some embodiments, the recombinant lysostaphin possesses a terminal cysteine. The present invention is not limited by the number of water-soluble polymers (e.g., PEGs) attached to a lysostaphin molecule. In some embodiments, a single water-soluble polymer is attached to a lysostaphin molecule. In some embodiments, two, three, four, five or more water-soluble polymers (e.g., PEGs) are attached to a lysostaphin molecule. In some embodiments, the conjugate comprises from one to about four polymer molecules per molecule of lysostaphin or lysostaphin analogue. In some embodiments, the PEG-lysostaphin conjugate or PEG-lysostaphin analogue conjugate has a mixed degree of conjugation. In some embodiments, the PEG-lysostaphin conjugate or PEG-lysostaphin analogue conjugate is a fractionated conjugate.

In some embodiments, the present invention provides a pharmaceutical composition for treating microbial infection comprising polyethylene glycol (PEG) conjugated to lysostaphin or a lysostaphin analogue, wherein the conjugate comprises a degradable linkage (e.g., an ester linkage), wherein at least a portion of the antimicrobial activity of the lysostaphin or lysostaphin analogue is retained, and a pharmaceutically acceptable carrier. In some embodiments, the lysostaphin or lysostaphin analogue conjugated to the polymer is less immunogenic than non-conjugated lysostaphin or lysostaphin analogue. In some embodiments, the lysostaphin or lysostaphin analogue conjugated to the polymer has a greater half-life and serum concentration than non-conjugated lysostaphin or lysostaphin analogue. In some embodiments, the lysostaphin or lysostaphin analogue is better able to penetrate tissue to access the point of bacterial infection (e.g., muscle infections or tissue abscesses). In some embodiments, the lysostaphin or lysostaphin analogue is capable of cleaving the cross-linked polyglycine bridges in the cell wall peptidoglycan of staphylococci. In some embodiments, the pharmaceutical composition comprises a non-conjugated antibacterial enzyme. The present invention is not limited by the type of antibacterial enzyme utilized in a pharmaceutical composition. Indeed, a variety of antibacterial enzymes are contemplated to be useful including, but not limited to, lysostaphin, lysostaphin analogue, lysozyme, mutanolysin, cellozyl muramidase, and combinations thereof. In some embodiments, the composition further comprises an antibiotic. The present invention is not limited by the type of antibiotic utilized in a pharmaceutical composition. Indeed, a variety of antibiotics are contemplated to be useful including, but not limited to, β-lactams, cephalosporins, aminoglycosides, sulfonamides, antifolates, macrolides, quinolones, glycopeptides, polypeptides and combinations thereof.

In some embodiments, the present invention provides a method for the prophylactic or therapeutic treatment of a microbial infection or colonization in a subject comprising administering to the subject a pharmaceutical composition comprising polyethylene glycol (PEG) conjugated to lysostaphin or a lysostaphin analogue, wherein the conjugate comprises a degradable linkage (e.g., an ester linkage), wherein at least a portion of the antimicrobial activity of the lysostaphin or lysostaphin analogue is retained, and a pharmaceutically acceptable carrier, in an amount effective for preventing or treating the infection. In some embodiments, the infection or colonization is a bacterial infection or colonization. In some embodiments, the bacterial infection or colonization is caused by bacteria from the genus Staphylococcus. In some embodiments, the bacteria comprises S. aureus. In some embodiments, the bacteria comprises S. epidermidis. In some embodiments, the colonization is colonization of the anterior nares, perineum, axilla, or other mucosal or skin surfaces. In some embodiments, the lysostaphin conjugated to PEG retains activity (e.g., in vivo) for a longer period of time than in the absence of conjugation to said PEG

### DESCRIPTION OF THE DRAWINGS

FIG. 1 depicts the lysis activity of lysostaphin conjugates according to the present invention in samples of heat-killed S. aureus capsule type 5;

FIG. 2 depicts the killing activity of lysostaphin conjugates according to the present invention in a high innoculum of live S. aureus capsule type 5;

FIG. 3 is a lysostaphin-capture immunoassay depicting the ability of PEG to shield lysostaphin from antibodies;

FIG. 4 depicts the serum concentrations and half-life of one lysostaphin conjugate at two different concentrations according to the present invention, in comparison to unconjugated lysostaphin.

FIG. 5 depicts the S. aureus capsule type 5 killing activity in saline of lysostaphin conjugates according to the present invention;

FIG. 6 depicts the S. aureus capsule type 5 killing activity in blood of lysostaphin conjugates according to the present invention;

FIG. 7 is an ELISA depicting the reactivity of anti-lysostaphin antibodies to lysostaphin conjugates according to the present invention;

FIG. 8 depicts the lysis activity of lysostaphin conjugates according to another aspect of the present invention in samples of heat-killed S. aureus capsule type 5;

FIG. 9 depicts the lysis activity of lysostaphin conjugates according to yet another aspect of the present invention in samples of heat-killed S. aureus capsule type 5.

FIG. 10 depicts the S. aureus capsule type 5 killing activity in saline of lysostaphin conjugates according to another aspect of the present invention;

FIG. 11 is an ELISA depicting the reactivity of anti-lysostaphin antibodies to lysostaphin conjugates according to another aspect of the present invention;

FIG. 12 depicts serum concentrations and half-life of lysostaphin conjugates according to another aspect of the present invention;

FIG. 13 compares the S. aureus capsule type 5 killing activity in saline of two different molecular weight lysostaphin conjugates according to the present invention; and

FIG. 14 depicts the S. aureus capsule type 5 killing activity in saline of a lysostaphin conjugate according to yet another aspect of the present invention.

FIG. 15 shows controlled release of native lysostaphin from a cleavable PEG-lysostaphin conjugate.

FIG. 16 shows an data from an ELISA measuring anti-lysostaphin antibody binding to native lysostaphin versus PEG-conjugated lysostaphin. Circles represent antibody binding to native lysostaphin whereas squares represent antibody binding to PEG-conjugated lysostaphin.

FIG. 17 shows serum pharmacokinetics of a cleavable PEG-lysostaphin conjugate.

FIG. 18 shows the efficacy of a cleavable PEG-lysostaphin conjugate in a mouse systemic S. aureus infection.

FIG. 19 shows the efficacy of a cleavable PEG-lysostaphin conjugate in a mouse thigh-abscess S. aureus infection.

Definitions

As used herein, the term “subject” refers to an individual (e.g., human, animal, or other organism) to be treated by the methods or compositions of the present invention. Subjects include, but are not limited to, mammals (e.g., murines, simians, equines, bovines, porcines, canines, felines, and the like), and most preferably includes humans. In the context of the invention, the term “subject” generally refers to an individual who will receive or who has received treatment for a condition characterized by the presence of bacteria (e.g., pathogenic bacteria such as methicillin-resistant S. aureus (MRSA)), or in anticipation of possible exposure to bacteria. As used herein, the terms “subject” and “patient” are used interchangeably, unless otherwise noted.

The term “diagnosed,” as used herein, refers to the recognition of a disease (e.g., caused by the presence of pathogenic bacteria) by its signs and symptoms (e.g., resistance to conventional therapies), or genetic analysis, pathological analysis, histological analysis, and the like.

As used herein the term, “in vitro” refers to an artificial environment and to processes or reactions that occur within an artificial environment. In vitro environments include, but are not limited to, test tubes and cell cultures. The term “in vivo” refers to the natural environment (e.g., an animal or a cell) and to processes or reaction that occur within a natural environment.

As used herein, the terms “attenuate” and “attenuation” used in reference to a feature (e.g. growth) of a bacterial cell or a population of bacterial cells refers to a reduction, inhibition or elimination of that feature, or a reducing of the effect(s) of that feature.

As used herein, the term “effective amount” refers to the amount of a composition (e.g., a composition comprising a water-soluble polymer conjugated lysostaphin (e.g., PEG-conjugated lysostaphin) sufficient to effect a beneficial or desired result (e.g., bacterial cell killing or a reduction in the number of infecting or colonizing bacteria). An effective amount can be administered in one or more administrations, applications or dosages and is not intended to be limited to a particular formulation or administration route.

As used herein, the term “administration” refers to the act of giving a drug, prodrug, pharmaceutical composition, or other agent, or therapeutic treatment (e.g., a composition of the present invention) to a physiological system (e.g., a subject or in vivo, in vitro, or ex vivo cells, tissues, and organs). Exemplary routes of administration to the human body can be through the eyes (ophthalmic), mouth (oral), skin (transdermal), nose (nasal), lungs (inhalant), mucosal (e.g., oral mucosa or buccal), rectal, ear, by injection (e.g., intravenously, subcutaneously, intratumorally, intraperitoneally, etc.) and the like.

As used herein, the term “treating a surface” refers to the act of exposing a surface to one or more compositions of the present invention. Methods of treating a surface include, but are not limited to, spraying, misting, submerging, and coating. Surfaces include organic surfaces (e.g., food products, surfaces of animals (e.g., skin, mucosa, etc.) and inorganic surfaces (e.g., medical devices, countertops, clothing, etc.).

As used herein, the term “co-administration” refers to the administration of at least two agent(s) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), or when a target of treatment (e.g., bacteria) have become less sensitive (e.g., resistant) to treatment with one or more agents administered alone (e.g., that when combined with one or more other agents, such targets of treatment display increased sensitivity (e.g., are non-resistant).

As used herein, the term “toxic” refers to any detrimental or harmful effects on a subject, a cell, or a tissue as compared to the same cell or tissue prior to the administration of the toxicant.

As used herein, the term “degradable linkage,” when used in reference to a polymer (e.g., PEG)-lysostaphin conjugate of the present invention, refers to a conjugate that comprises a physiologically cleavable linkage (e.g., a linkage that can be hydrolyzed (e.g., in vivo) or otherwise reversed (e.g., via enzymatic cleavage)). Such physiologically cleavable linkages include, but are not limited to, ester, carbonate ester, carbamate, sulfate, phosphate, acyloxyalkyl ether, acetyl, and ketal linkages (See, e.g., U.S. Pat. No. 6,838,076, herein incorporated by reference in its entirety). Similarly, the conjugate may comprise a cleavable linkage present in the linkage between the polymer and lysostaphin, or, may comprise a cleavable linkage present in the polymer itself (e.g., such that when cleaved, a small portion of the polymer remains on the lysostaphin molecule) (See, e.g., U.S. Pat. App. Nos. 20050158273 and 20050181449, each of which is herein incorporated by reference in its entirety). For example, a PEG polymer comprising an ester linkage can be utilized for conjugation to lysostaphin to create a PEG-lysostaphin conjugate (See, e.g., Kuzlowski et al., Biodrugs, 15, 419-429 (2001). A conjugate that comprises a degradable linkage of the present invention is capable of generating lysostaphin that is free (e.g., completely or partially free) of the polymer (e.g., in vivo after hydrolysis of the linkage).

As used herein, the term “pharmaceutical composition” refers to the combination of an active agent (e.g., a water-soluble polymer conjugated lysostaphin (e.g., PEG-conjugated lysostaphin)) with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions (e.g., toxic, allergic, or immunological reactions) when administered to a subject.

As used herein, the term “topically” refers to application of the compositions of the present invention to the surface of the skin and/or mucosal cells and tissues (e.g., alveolar, buccal, lingual, masticatory, vaginal, or nasal mucosa, and other tissues and cells that line hollow organs or body cavities).

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintrigrants (e.g., potato starch or sodium starch glycolate), and the like. The compositions also may include stabilizers and preservatives. Examples of carriers, stabilizers, and adjuvants are described in the art (See e.g., Martin, Remington's Pharmaceutical Sciences, 15th Ed., Mack Publ. Co., Easton, Pa. (1975), incorporated herein by reference).

As used herein, the term “medical devices” includes any material or device that is used on, in, or through a subject's or patient's body, for example, in the course of medical treatment (e.g., for a disease or injury). Medical devices include, but are not limited to, such items as medical implants, wound care devices, drug delivery devices, and body cavity and personal protection devices. The medical implants include, but are not limited to, urinary catheters, intravascular catheters, dialysis shunts, wound drain tubes, skin sutures, vascular grafts, implantable meshes, intraocular devices, heart valves, and the like. Wound care devices include, but are not limited to, general wound dressings, biologic graft materials, tape closures and dressings, and surgical incise drapes. Drug delivery devices include, but are not limited to, needles, drug delivery skin patches, drug delivery mucosal patches and medical sponges. Body cavity and personal protection devices, include, but are not limited to, tampons, sponges, surgical and examination gloves, and toothbrushes. Birth control devices include, but are not limited to, intrauterine devices (IUDs), diaphragms, and condoms.

As used herein, the term “therapeutic agent” refers to a composition that decreases the infectivity, colonization, morbidity, or onset of mortality in a subject contacted by a pathogenic microorganism or that prevent infectivity, morbidity, or onset of mortality in a host contacted by a pathogenic microorganism. Therapeutic agents encompass agents used prophylactically (e.g., in the absence of a pathogen) in view of possible future exposure to a pathogen. Such agents may additionally comprise pharmaceutically acceptable compounds (e.g., adjuvants, excipients, stabilizers, diluents, and the like). In some embodiments, the therapeutic agents of the present invention are administered in the form of topical compositions, injectable compositions, ingestible compositions, and the like. When the route is topical, the form may be, for example, a solution, cream, ointment, salve or spray.

As used herein, the term “pathogen” refers a biological agent that causes a disease state (e.g., infection, sepsis, etc.) in a host. “Pathogens” include, but are not limited to, viruses, bacteria, archaea, fungi, protozoans, mycoplasma, prions, and parasitic organisms.

The terms “bacteria” and “bacterium” refer to all prokaryotic organisms, including those within all of the phyla in the Kingdom Procaryotae. It is intended that the term encompass all microorganisms considered to be bacteria including Mycoplasma, Chlamydia, Actinomyces, Streptomyces, and Rickettsia. All forms of bacteria are included within this definition including cocci, bacilli, spirochetes, spheroplasts, protoplasts, etc. Also included within this term are prokaryotic organisms that are Gram-negative or Gram-positive. In some embodiments, bacteria are continuously cultured. In some embodiments, bacteria are uncultured and existing in their natural environment (e.g., at the site of a wound or infection) or obtained from patient tissues (e.g., via a biopsy). Bacteria may exhibit pathological growth or proliferation. Examples of bacteria include, but are not limited to, bacterial cells of a genus of bacteria selected from the group comprising Salmonella, Shigella, Escherichia, Enterobacter, Serratia, Proteus, Yersinia, Citrobacter, Edwardsiella, Providencia, Klebsiella, Hafnia, Ewingella, Kluyvera, Morganella, Planococcus, Stomatococcus, Micrococcus, Staphylococcus, Vibrio, Aeromonas, Plessiomonas, Haemophilus, Actinobacillus, Pasteurella, Mycoplasma, Ureaplasma, Rickettsia, Coxiella, Rochalimaea, Ehrlichia, Streptococcus, Enterococcus, Aerococcus, Gemella, Lactococcus, Leuconostoc, Pedicoccus, Bacillus, Corynebacterium, Arcanobacterium, Actinomyces, Rhodococcus, Listeria, Erysipelothrix, Gardnerella, Neisseria, Campylobacter, Arcobacter, Wolinella, Helicobacter, Achromobacter, Acinetobacter, Agrobacterium, Alcaligenes, Chryseomonas, Comamonas, Eikenella, Flavimonas, Flavobacterium, Moraxella, Oligella, Pseudomonas, Shewanella, Weeksella, Xanthomonas, Bordetella, Franciesella, Brucella, Legionella, Afipia, Bartonella, Calymmatobacterium, Cardiobacterium, Streptobacillus, Spirillum, Peptostreptococcus, Peptococcus, Sarcinia, Coprococcus, Ruminococcus, Propionibacterium, Mobiluncus, Bifidobacterium, Eubacterium, Lactobacillus, Rothia, Clostridium, Bacteroides, Porphyromonas, Prevotella, Fusobacterium, Bilophila, Leptotrichia, Wolinella, Acidaminococcus, Megasphaera, Veilonella, Norcardia, Actinomadura, Norcardiopsis, Streptomyces, Micropolysporas, Thermoactinomycetes, Mycobacterium, Treponema, Borrelia, Leptospira, and Chlamydiae.

As used herein, the term “microorganism” refers to any species or type of microorganism, including but not limited to, bacteria, archaea, fungi, protozoans, mycoplasma, and parasitic organisms.

As used herein, the term “non-human animals” refers to all non-human animals including, but not limited to, vertebrates such as rodents, non-human primates, ovines, bovines, ruminants, lagomorphs, porcines, caprines, equines, canines, felines, aves, etc.

As used herein, the term “kit” refers to any delivery system for delivering materials. In the context of reaction materials (e.g., compositions comprising a water-soluble polymer conjugated lysostaphin (e.g., PEG-conjugated lysostaphin)), such delivery systems include systems that allow for the storage, transport, or delivery of reaction reagents and/or supporting materials (e.g., written instructions for using the materials, etc.) from one location to another. For example, kits include one or more enclosures (e.g., boxes) containing the relevant reaction reagents and/or supporting materials. As used herein, the term “fragmented kit” refers to delivery systems comprising two or more separate containers that each contain a subportion of the total kit components. The containers may be delivered to the intended recipient together or separately. For example, a first container may contain a composition comprising a water-soluble polymer conjugated lysostaphin (e.g., PEG-conjugated lysostaphin) for a particular use, while a second container contains a second agent (e.g., an antibiotic or spray applicator). Indeed, any delivery system comprising two or more separate containers that each contains a subportion of the total kit components are included in the term “fragmented kit.” In contrast, a “combined kit” refers to a delivery system containing all of the components of a reaction materials needed for a particular use in a single container (e.g., in a single box housing each of the desired components). The term “kit” includes both fragmented and combined kits.

### DETAILED DESCRIPTION OF THE INVENTION

The present invention relates to the conjugation of antimicrobial agents to water-soluble polymers to improve their clinical properties in terms of their pharmacokinetics, pharmacodynamics, and reduced immunogenicity. More specifically, the present invention relates to the conjugation of antimicrobial agents such as lysostaphin to poly(alkylene oxides) (e.g., polyethylene glycol (PEG)).

PEGylation is a process in which therapeutic polypeptides, such as enzymes and hormones, are coupled to (e.g., conjugated to) one or more chains of polyethylene glycol to provide improved clinical properties in terms of pharmacokinetics, pharmacodynamics, and immunogenicity.

PEGylation can alter the characteristics of the polypeptide without affecting the ability of the parent molecule to function, thereby producing a physiologically active, reduced or non-immunogenic, water-soluble polypeptide composition. The polymer protects the polypeptide from loss of activity by reducing its clearance and susceptibility to enzymatic degradation, and the composition can be injected into the mammalian circulatory system with reduced immunogenic response. PEGylation of enzymes and other polypeptides is described in detail in U.S. Pat. No. 4,179,337 to Davis et al., and in Zalipsky, “Functionalized Poly(ethylene glycol) for Preparation of Biologically Relevant Conjugates,” Bioconjugate Chem., 6, 150-165 (1995), both of which are incorporated by reference in their entirety herein.

Davis et al. disclose that polypeptides modified with PEG have dramatically reduced immunogenicity and antigenicity. PEG conjugates exhibit a wide range of solubilities and low toxicity, and have been shown to remain in the bloodstream considerably longer than the corresponding native compounds, yet are readily excreted.

PEG conjugation is typically accomplished by means of two commonly used types of linkages. One type of conjugation reacts a polypeptide amino group with a PEG molecule having an active carbonate, ester, aldehyde or tresylate group. Another type of conjugation reacts a polypeptide thiol group with a PEG molecule having an active vinyl sulfone, maleimide, haloacyl or thiorthopyridyl group, or other suitable electrophile (See, e.g., Hermanson, Bioconjugate Techniques (Academic Press, San Diego 1966). One of the two terminal hydroxyls of the PEG is blocked by conversion to an alkoxy group when intermolecular cross-linking is not desired. A PEG molecule with one terminal methoxy group is referred to as mPEG.

The PEG molecule may be linear or branched, whereby PEG conjugates can be created by conjugating a single large PEG moiety to a single conjugation site, a single branched (e.g., smaller) PEG moiety to a single conjugation site, or several small PEG moieties to multiple conjugation sites. When multiple conjugation sites are employed, this can result in the loss of bioactivity. In addition to PEG homopolymers, the polymer molecule can be copolymerized with other alkylene oxide moieties, or it can be another poly(alkylene oxide) homopolymer or copolymer.

In some embodiments, PEG polymer comprising an ester linkage is utilized for conjugation to lysostaphin to create a PEG-lysostaphin conjugate. For example, a PEG polymer synthesized by reacting mPEG with succinic anhydride, followed by activation of the carboxylic acid as the succinimidyl ester can be used. Contained in the structure of this polymer is a backbone that includes an ester linkage. This linkage remains after the mPEG is coupled to lysostaphin (e.g., via an amide linkage) and is susceptible to hydrolysis after the polymer conjugate has been attached to the protein (See, e.g., Kuzlowski et al., Biodrugs, 15, 419-429 (2001). Thus, in some embodiments, a PEG-lysostaphin conjugate is a degradable conjugate (e.g., the conjugate can be hydrolyzed (e.g., in vivo) to provide lysostaphin free of PEG).

In some embodiments, the chemical linkage between lysostaphin and the polymer (e.g., the polymer branch point) may be degradable (e.g., hydrolytically unstable). Alternatively, one or more degradable linkages may be contained in the polymer backbone to allow generation in vivo of a PEG-lysostaphin conjugate having a smaller PEG chain than in the initially administered conjugate. Such optional features of the polymer conjugate may provide for additional control over the final desired pharmacological properties of the conjugate upon administration. For example, a large and relatively inert conjugate (e.g., having one or more high molecular weight PEG chains attached thereto (e.g., one or more PEG chains having a molecular weight greater than about 10,000, wherein the conjugate possesses essentially no bioactivity)) may be administered, which then is hydrolyzed to generate a bioactive conjugate possessing a portion of the originally present PEG chain. In this way, the properties of the PEG-lysostaphin conjugate may be somewhat more effectively tailored. For example, absorption of the initial polymer conjugate may be slow upon initial administration. Upon in-vivo cleavage of the hydrolytically degradable linkage, either free lysostaphin (depending upon the position of the degradable linkage) or lysostaphin having a small polyethylene tag attached thereto, is then released and more readily absorbed and/or circulated in the blood.

In some embodiments, the intact polymer-conjugate, prior to hydrolysis, is minimally degraded upon administration, such that hydrolysis of the cleavable bond is effective to govern the slow rate of release of lysostaphin (e.g., into the bloodstream), as opposed to enzymatic degradation of lysostaphin prior to its release into the systemic circulation.

Degradable linkages (e.g., physiologically cleavable linkages) include, but are not limited to, ester, carbonate ester, carbamate, sulfate, phosphate, acyloxyalkyl ether, acetyl, and ketal. Such conjugates should possess a physiologically cleavable bond that is stable upon storage and upon administration. For instance, a PEG-lysostaphin conjugate with a degradable linkage maintains its integrity upon manufacturing of the final pharmaceutical composition, upon dissolution in an appropriate delivery vehicle, if employed, and upon administration irrespective of route.

In some embodiments, PEG-lysostaphin conjugates having biodegradable linkages and useful in the present invention are represented by the following structures: PEG1-W-PEG2-lysostapin (where PEG1 and PEG2 can be the same or different) or PEG-W-lysostaphin wherein W represents a weak, biodegradable linkage (e.g., a hydrolytically cleavable linkage (e.g., an ester linkage)). These conjugates contain PEG arms or portions of PEG arms that are removable (e.g., cleavable) in-vivo. These PEG-lysostaphin conjugates may be substantially biologically inactive when intact, either due to the size of the intact PEG-portion of the molecule or due to steric blockage of the active sites on the lysostaphin molecule by the PEG chain. Alternatively, the PEG-lysostaphin conjugates may be biologically active when intact and remain so when the conjugate (e.g., conjugate linkage) is degraded (e.g., providing lysostaphin (e.g., lysostaphin free of PEG or retaining a small portion of the PEG molecule). In some embodiments, PEG-lysostaphin conjugates (e.g., possessing degradable linkages) exhibit a greater amount of intracellular (e.g., within phagocytes) activity (e.g., killing activity) than lysostaphin that is not so conjugated. Although an understanding of the mechanism is not necessary to practice the present invention and the present invention is not limited to any particular mechanism of action, in some embodiments, a PEG-lysostaphin conjugate exhibits greater intracellular activity (e.g., within a phagocytic cell) than lysostaphin not so conjugated because the PEG protects the conjugate from degradation (e.g., by various proteases) within cells.

For purposes of the present invention, the term “antimicrobial agent” is defined as including any substances (e.g., chemical, protein, peptide or lipopeptide, including enzymes) that, upon contact (e.g., in a host subject), kill microbes or inhibit microbe metabolism without damaging the surrounding environment (e.g., host cells or tissues), or upon contact with a host, elicit a harmful host response. This includes substances that would without polymer conjugation otherwise damage host cells or tissues or elicit a harmful response.

The term “lysostaphin” includes wild type lysostaphin (e.g., isolated and/or purified from the natural lysostaphin producing bacterial strains) and any lysostaphin mutant or variant, any recombinant, or related enzyme, or any synthetic version or fragment of lysostaphin that retains the proteolytic activity, in vivo and in vitro, to cleave cross-linked polyglycine bridges (e.g., in cell wall peptidoglycan (e.g., of staphylococci)). Variants may be generated by post-translational processing of the protein (e.g., by enzymes present in a producer strain or by means of enzymes or reagents introduced at any stage of a manufacturing process) or by mutation of the structural gene. Mutations may include site deletion, insertion, domain removal and replacement mutations.

The term “lysostaphin analogue” is defined as including any form of lysostaphin that is not wild-type pro-lysostaphin or mature lysostaphin. The lysostaphin and lysostaphin analogues contemplated in the present invention may be recombinantly expressed (e.g., from a cell culture or higher recombinant species such as a mouse or otherwise, expressed in mammalian cell hosts, insects, bacteria, yeast, reptiles, fungi, etc.), or synthetically constructed. This includes the activity retaining synthetic construction including synthetic peptides and polypeptides or recombinant expression of portions of the lysostaphin polypeptide responsible for its activity against staphylococci alone, or as part of a larger protein or polypeptide, including chimeric proteins, containing the active site(s) of one or more other antimicrobial proteins or peptides that are active against staphylococci, or against one or more other microbe(s) or bacteria species to provide a broader spectrum of activity. These analogs include polypeptides which have either the staphylococcal binding activity or the polyglycine cleavage activity of lysostaphin or both activities.

Lysostaphin is naturally produced by bacteria as a pro-enzyme that is cleaved to produce a fully active mature form of lysostaphin (e.g., with the propeptide and leader sequence removed). Recombinant or synthetically produced lysostaphin preparations can be used that contain only a fully-active mature form of lysostaphin. The recombinant expression of homogenous lysostaphin, and homogenous fully active lysostaphin-containing compositions prepared from the expressed protein are disclosed in U.S. Patent App. Pub. No. 20050118159, the disclosure of which is incorporated herein by reference in its entirety.

Antimicrobial agents such as lysostaphin and lysostaphin analogue proteins described herein may be conjugated to water-soluble polymers via free amino groups, either at lysine or arginine residues or a free amino group, if any, at the N-terminus. Other suitable antimicrobial agents include nisin, amphotericin-alpha, and the like.

From a minimum of one up to about twelve water-soluble polymer molecules can be attached to each molecule of an antimicrobial agent (e.g., lysostaphin), although, in some embodiments, more than 12 polymers may be conjugated to a single lysostaphin molecule. Because one object of the modification is to increase in vivo half life over the unconjugated antimicrobial agents with reduced immunogenicity, the number of conjugated polymers and the weight-average molecular weight of these molecules can be selected to provide a polymer conjugate of an antimicrobial agent with an apparent weight-average molecular weight from about 5 to 40 kDa, up to about 200 kDa. However, the present invention is not limited by any particular weights. For example, a conjugate may have a higher (e.g., greater than 200 kDa) or lower (less than 5 kDa) molecular weight.

Poly(alkylene oxides), when used, typically have weight-average molecular weights between about 1 kDa and about 100 kDa, more typically between about 2, 3 or 4 and about 50 kDa, and also between about 5 or 10 and about 40 kDa, depending upon the number of conjugates per lysostaphin molecule. When conjugated to lysostaphin, from 1 to about 10 poly(alkylene oxide) molecules per lysostaphin molecule can be used, with from 1 to about 3 or 4 being typically used, and 1 or 2 being more typical. However, a lysostaphin conjugate may display a weight greater than 100 kDa and/or may be conjugated to more than 10 poly(alkylene oxide) molecules. Similarly, a lysostaphin molecule may be conjugated to 2 or more different types of poly(alkylene oxide) molecules. Lysostaphin compositions with mixed degrees of conjugation may also be used, or the lysostaphin conjugate may be fractionated so that a lysostaphin conjugate is obtained that essentially consists of a fraction of lysostaphin conjugated to essentially the same number of polymers. For example, essentially all lysostaphin in a fractionated sample is conjugated to 1, 2, 3 or more polymers, but not mixtures thereof.

When poly(alkylene oxides) are used, they may be straight chained or branched. Although an understanding of the mechanism is not necessary to practice the present invention and the present invention is not limited to any particular mechanism of action, branched poly(alkylene oxides), such as branched PEG, because of their larger spatial volume, are less likely to penetrate protein crevasses, which are often the binding motifs and active sites of enzymes. Typical poly(alkylene oxides) consist of C₂-C₄ alkylene oxide groups, separately as homopolymers or in combination. This includes PEGs, poloxamers and poloxamines. The poly(alkylene oxides) can be substituted at one end with an alkyl group, or it may be unsubstituted. The alkyl group, when present, can be a C₁-C₄ alkyl group, and is typically a methyl group.

Suitable covalent modification reactions are well known and essentially conventional. Generally the process involves preparing an activated polymer and thereafter reacting the antimicrobial agent with the activated polymer. The reaction using N-hydroxysuccinimide activated MPEG (mPEG-NHS) described by Davis et al. can be used. MPEG-NHS is commercially available from Shearwater Corp. of Huntsville, Ala., now known as NEKTAR Therapeutics, AL.

Typically, the reaction is carried out in a buffer of pH about 7-8, frequently at about 10 mM Hepes pH 7.5, 100 mM NaCl. The reaction is carried out generally at 0° to about 25° C. for from about 20 minutes to about 12 hours, for example, for 25-35 minutes at about 20° C. or three hours at 4° C. Following the conjugation, the desired product can be recovered and purified (e.g., by column chromatography and the like).

The antimicrobial agent thus modified is then formulated as either an aqueous solution, semi-solid formulation, or dry preparation (e.g., lyophilized, crystalline or amorphous, with or without additional solutes for osmotic balance) for reconstitution. Formulations may be in, or reconstituted in, for example, a non-toxic, stable, pharmaceutically acceptable, aqueous carrier medium, at a pH of about 3 to 8, typically 5 to 8, for administration by conventional protocols and regimes or in a semi-solid formulation such as a cream. Delivery can be via, for example, ophthalmic administration, intravenous (iv), intramuscular, subcutaneous or intraperitoneal routes, intranasal, topical or intrathecally or by inhalation or used to coat medical devices, catheters and implantable devices, or by direct installation into an infected site so as to permit blood and tissue levels in excess of the minimum inhibitory concentration (MIC) of the active agent to be attained and thus to effect a reduction in microbial titers in order to cure, alleviate or prevent an infection or colonization. In some embodiments, the antimicrobial agent is formulated as a semi-solid formulation, such as a cream (e.g., that is used in a topical or intranasal formulation).

Furthermore, the antimicrobial conjugate can be co-administered, simultaneously or alternating, with other antimicrobial agents so as to more effectively treat an infectious disease. Formulations may be in, or be reconstituted in, semi-solid formulations for topical, ophthalmic, or intranasal application, liquids suitable for ophthalmic administration, bolus iv or peripheral injection or by addition to a larger volume iv drip solution, or may be in, or reconstituted in, a larger volume to be administered by slow iv infusion. For example, a lysostaphin conjugate can be administered in conjunction with antibiotics that interfere with or inhibit cell wall synthesis, such as penicillins, such as nafcillin, and other alpha- or beta-lactam antibiotics, cephalosporins such as cephalothin, aminoglycosides, sulfonamides, antifolates, macrolides, quinolones, glycopepetides such as vancomycin and polypeptides. In some embodiments, a lysostaphin conjugate is administered in conjunction with one or more antibiotics that inhibit protein synthesis (e.g., aminoglycosides such as streptomycin, tetracyclines, and streptogramins). In some embodiments, a lysostaphin conjugate is administered with monoclonal antibodies; other non-conjugated antibacterial enzymes such as lysostaphin, lysozyme, mutanolysin, and cellozyl muramidase; peptides (e.g., defensins); and lantibiotics (e.g., nisin); or any other lanthione-containing molecules (e.g., subtilin).

Agents co-administered with a lysostaphin conjugate may be formulated together with the lysostaphin conjugate as a fixed combination or may be used extemporaneously in whatever formulations are available and practical and by whatever routes of administration are known to provide adequate levels of these agents at the sites of infection.

In preferred embodiments, conjugates according to the present invention possess at least a portion of the anti-microbial activity of the corresponding non-conjugated antimicrobial agent. A conjugate of the present invention may be administered in increased dosages and/or at less frequent intervals due to the decreased immunogenicity and increased circulating half-life produced by conjugation to a water soluble polymer (e.g., PEG). In some embodiments, a conjugate retains at least 10% of the activity of the non-conjugated antimicrobial agent. In some embodiments, a conjugate retains at least 20% of the activity of the non-conjugated antimicrobial agent. In some embodiments, a conjugate retains at least 30% of the activity of the non-conjugated antimicrobial agent. In some embodiments, a conjugate retains at least 40% of the activity of the non-conjugated antimicrobial agent. In some embodiments, a conjugate retains at least 50% of the activity of the non-conjugated antimicrobial agent. In some embodiments, a conjugate retains at least 60% of the activity of the non-conjugated antimicrobial agent. In some embodiments, a conjugate retains at least 70% of the activity of the non-conjugated antimicrobial agent. In some embodiments, a conjugate retains at least 80% of the activity of the non-conjugated antimicrobial agent. In some embodiments, a conjugate retains at least 90% of the activity of the non-conjugated antimicrobial agent. In some embodiments, a conjugate retains 90% or more (e.g., 95%, 97%, 99% or more) of the activity of the non-conjugated antimicrobial agent.

Suitable dosages and regimes of a lysostaphin conjugate may vary with the severity of the infection and the sensitivity of the infecting organism and, in the case of combination therapy, may depend on the particular agent (e.g., anti-staphylococcal agent) co-administered. Dosages may range from about 0.05 to about 500 mg/kg/day (e.g., in some embodiments, range from 0.1-10 mg/kg/day, in some embodiments, range from 10-100 mg/kg/day, in some embodiments, range from 100-200 mg/kg/day, in some embodiments, range from 200-400 mg/kg/day, in some embodiments, range from 400-500 mg/kg/day), although higher or lower doses may be provided, given as single or divided doses, or given by continuous infusion.

The present invention is further illustrated by the following examples that teach those of ordinary skill in the art how to practice the invention. The following examples are merely illustrative of the invention and disclose various beneficial properties of certain embodiments of the invention. The following examples should not be construed as limiting the invention as claimed.

### EXPERIMENTAL

The following examples are provided in order to demonstrate and further illustrate certain preferred embodiments and aspects of the present invention and are not to be construed as limiting the scope thereof.

### Example 1

### Materials

As an example of an antimicrobial agent, lysostaphin was employed for these studies, which can be repeated with essentially any antimicrobial agent, as the term is defined by the present specification. Lysostaphin (AMBICIN L) was obtained from AMBI, Inc. (now Nutrition 21). The mPEG2-NHS esters, 10 and 40 kDa were purchased from Shearwater Corporation (Huntsville, Ala.) (now NEKTAR Therapeutics, AL). Sodium Borate, DMSO, bovine serum albumin, and extravidin-HRP were purchased from SIGMA Chemical Co. (St. Louis, Mo.). Glycine was purchased from EM Science (Gibbstown, N.J.). The NUPAGE Electrophoresis System and Colloidal Blue stain were purchased from INVITROGEN (Carlsbad, Calif.). SEPHACRYL S-100HR and HITRAP SP FF were purchased from AMERSHAM-PHARMACIA (Piscataway, N.J.). Tryptic Soy Broth, TSB, and Cation-Adjusted Mueller Hinton Broth, CAMHB, were purchased from BECTON DICKINSON (Sparks, Md.). TMB Microwell and 450 STOP Reagent were purchased from BIOFX (Owings Mills, Md.).

### Example 2

### Characterization of PEGylated Lysostaphin

Lysostaphin PEGylation. Lysostaphin at 0.27, 1, or 5 mg/mL was dissolved in either 0.2M borate buffer (pH 8.5) or DMSO. The mPEG2-NHS esters were prepared in DMSO and added to the lysostaphin solution in molar excess at ratios of 40, 20, 10, 5 or 2.5:1. PEGylation was performed with three different buffer conditions, all at room temperature for 1, 2, or 3 hours: borate buffer (with <10% DMSO contributed by adding PEG), 50% borate/50% DMSO, and 100% DMSO. All reactions were quenched by added glycine to 25 mM and vortexing.

PEG conjugation to lysostaphin was evaluated by SDS-PAGE with the NUPAGE Electrophoresis System. Non-reduced samples (300 ng) were run on a Novex 4-12% Bis-Tris gel at 115V and stained with colloidal blue. PEGylated lysostaphin was separated from unreacted lysostaphin by running the reaction mixture over a SEPHACRYL S-100HR column. Purified PEG-lysostaphin was concentrated and saved for activity assays.

Alternatively, unconjugated lysostaphin was removed from the sample by ion-exchange chromatography. Lysostaphin, but not PEG-lysostaphin, was bound onto a HITRAP SP FF column in 50 mM sodium phosphate buffer, pH 7.0. The column was washed with the same buffer until the OD280 of the eluate was reduced to background levels. Bound lysostaphin was then removed, and the column regenerated by washing with 50 mM sodium phosphate plus 1 M NaCl, pH 7.0. This process was repeated several times until the PEG-lysostaphin fraction (unbound) was at least 99% pure of unconjugated lysostaphin.

In Vitro Activity of PEG-Lysostaphin. Lysostaphin's ability to lyse staphylococcus aureus type-5 (SA5) was determined by measuring the drop in absorbance at 650 nm of a solution containing heat-killed SA5 (HKSA5). HKSA5 was prepared by incubating live bacteria at 62° C. for 2 hours and then diluted such that the initial absorbance was about 1. Lysostaphin was then added at a concentration of 32 μg/mL and absorbance readings were taken every 60 seconds for 20 minutes. Clearance of live SA5 cultures were measured by adding lysostaphin from 0 to 10 μg/mL to an SA5 suspension in PBS (% T=40). The samples were incubated at 37° C. for 1 hour and then spread onto blood agar plates. After overnight culture at 37° C., colonies were counted and compared to untreated samples.

The minimum inhibitory concentration (MIC) of the conjugated lysostaphin was determined against S. aureus strain SA5-Lab (a methicillin susceptible strain of S. aureus which expresses capsule type 5). After an overnight culture of SA5-Lab in TSB, the bacteria was diluted to % T=80. 100 μL of growth media (CAMHB+1% BSA+0 to 32 μg lysostaphin) was added to each well of a 96-well culture plate. 5 μL of SA5-Lab was added to each well and the plate incubated at 37° C. and 200 rpm for 24 hours. The absorbance at 650 nm was read and the MIC defined as the last well where there was no SA5-Lab growth.

Anti-Lysostaphin Binding Activity. A lysostaphin capture ELISA was performed to determine if PEGylated lysostaphin shields the protein from antibody binding. 96-well microtiter plates were coated with a polyclonal rabbit anti-lysostaphin antibody overnight. The wells were blocked with 1% BSA followed by incubation with the lysostaphin samples in PBS/0.5% Tween 20 plus 0.1% BSA. Lysostaphin binding was detected with biotin-labeled, polyclonal rabbit anti-lysostaphin followed by extravidin-HRP incubation and TMB colorimetric detection. The plates were measured at an absorbance of 450 nm in a SPECTRAMAX PLUS plate reader (Molecular Devices; Sunnyvale, Calif.).

Serum Pharmacokinetics of PEG-Lysostaphin. CF₁ mice were injected in the tail vein with S-100HR purified PEG-lysostaphin at a dose of 0.8 or 0.2 mg (4 or 1 mg/mL in 0.2 mL PBS). Control mice were injected with 0.8 mg of unconjugated lysostaphin. Blood was collected by orbital eye bleeding at 1, 4, 7, and 24 hours post-administration. The blood was incubated at 37° C. for 30 minutes followed by 4° C. for 30 minutes. Serum was then separated by centrifugation at 1000 g for 10 minutes. The serum concentration of lysostaphin was determined by ELISA as described above.

Results. Lysostaphin has a high net charge of +10.53 at pH 7 due to a large number of lysine (16) and arginine (6) residues. The primary amine groups of the side chains of these lysines are ideal targets to covalently link PEG that has been activated with N-hydroxysuccinimide. Branched PEG's were chosen because their larger spatial volume makes them less likely to penetrate protein crevasses, often the binding motifs and active sites of enzymes.

The reaction conditions can be manipulated to create a PEGylated lysostaphin molecule that has an optimal balance between enzyme activity and enhanced properties such as reduced immunogenicity, decreased antibody binding and toxicity and increased serum half-life and efficacy. Unique reaction groups may be added to lysostaphin in order to conjugate PEG in a number controlled and site specific manner. For example, the creation of sulfhydryl groups is one way to achieve this goal because lysostaphin does not contain any cysteine residues. Another way to achieve this goal is to introduce thiol groups into the protein by introducing the thiol-containing amino acid cysteine into the amino acid sequence of the protein through genetic engineering.

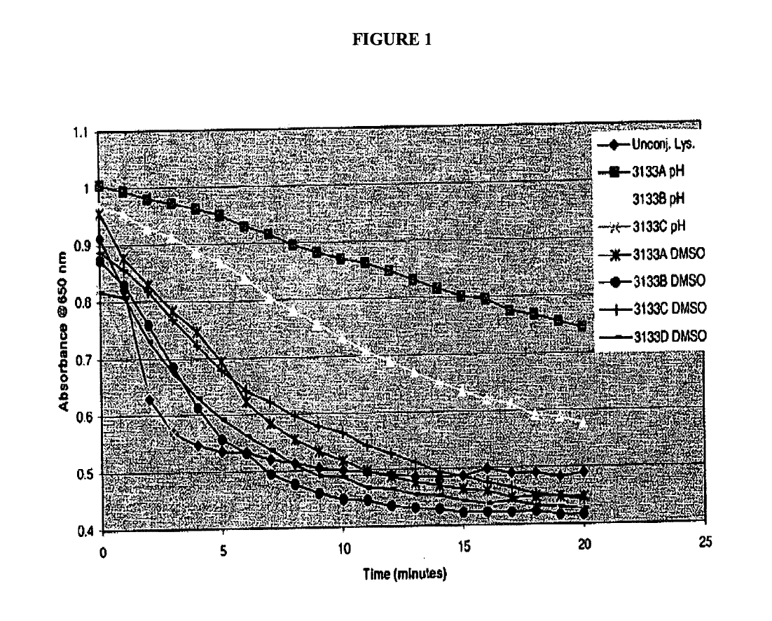

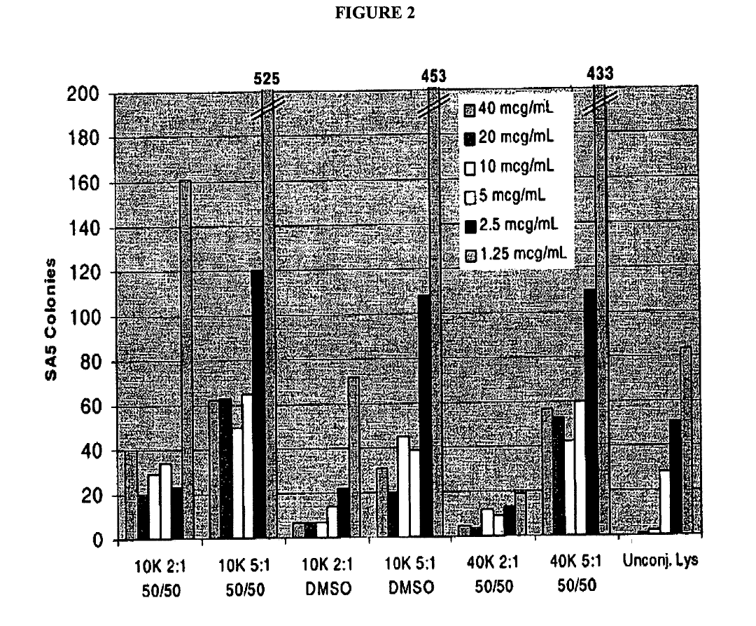

Enzyme Killing Activity on SA5-Lab. The in vitro killing activity of five PEGylated lysostaphin samples was tested by measuring lysis of heat-killed SA5-Lab (See FIG. 1) and killing of live SA5 (See FIG. 2). In FIG. 1, lysostaphin conjugated to 10 kDa PEG is represented in lines 3133A-B pH and 3133A-DMSO. The pH designation indicates PEGylation reactions that took place in aqueous solution. The DMSO designation designates PEGylation reactions that were performed in 100% DMSO. All samples were positive for enzyme activity. A reduction in activity with increasing degrees of PEGylation indicated that the 10 kDa PEG is small enough to gain access to lysostaphin's active site or its peptido-glycan binding domain, thus reducing its enzyme activity. In contrast, no reduction in enzyme activity was observed with the 40 kDa PEG samples despite the fact that no size separation was performed on these samples. This indicates that the highly PEGylated forms retain similar activity to lightly conjugated forms and indicates that the 40 kDa PEG cannot easily access sites important for enzyme function.

The ability to kill live SA5-Lab in vitro was tested with decreasing concentrations of PEGylated lysostaphin (See FIG. 2). As was observed for the heat-killed assay, higher degrees of PEG conjugation reduced lysostaphin activity and the 40 KDa conjugate retained more activity than the 10 kDa form. However, new properties of PEG-lysostaphin emerged that were not apparent in the heat-killed assay. The killing curve for unconjugated lysostaphin shows the titration of response with decreasing enzyme concentrations, but all of the PEGylated enzymes appear to have a flat response over the first four dilutions before finally titrating upward. In particular, the 40 kDa PEG at a 2:1 ratio maintains greater killing over the lowest three concentrations, compared to unconjugated lysostaphin, but is comparatively less active at the three highest concentrations. This finding indicates that PEGylated lysostaphin has modified activity or metabolism. PEG may shield the enzyme from degradative proteases that are released from the bacteria as they are killed, thus enabling lysostaphin to remain active for a longer period of time at lower concentrations. Another possibility is that PEG conjugated onto lysostaphin may alter the enzymes interaction with the bacterial cell wall. Reduced binding affinity to its cell wall docking site, while still enabling peptidoglycan cleavage, would result in quicker enzyme release and speed the recycling of lysostaphin for the next round of cleavage. Either of these explanations, and others yet undiscovered, could explain the observed response and each is equally encouraging for the prospect of creating a PEGylated form of lysostaphin that is superior to an un-conjugated form.

Inhibition of SA5-Lab Growth. The minimum inhibitory concentration (MIC) is a quantitative measure of a drug's activity that is typically used to examine levels of resistance in different bacterial strains. This assay was used against a single strain of SA5-Lab to measure loss of drug activity upon lysostaphin PEGylation, as shown in Table 1 (units for the second column is μg/ml). Several formulations retained high levels of activity although none were as high as un-conjugated lysostaphin. The pattern of activity observed with the different PEG-lysostaphin species is consistent with that observed in the previous killing assays. The more lightly PEGylated lysostaphin retained greater activity than highly conjugated forms and under the same reaction conditions, the 40 kDa PEG conjugate was eight times more active than the 10 kDa PEG conjugate.

TABLE 1
MIC of PEGylated Lysostaphin Against SA5

| TYPE OF LYSOSTAPHIN | MIC μg/ml |
|---|---|
| 10K 2:1 50/50 | 4 |
| 10K 5:1 50/50 | >32 |
| 10K 2:1 DMSO | 2 |
| 10K 5:1 DMSO | 16 |
| 40K 2:1 50/50 | 0.5 |
| 40K 2:1 50/50 | >32 |
| Unconjugated | 0.13 |

Both of these findings support the conclusion that low degrees of PEGylation result in an active enzyme and that the bulkier 40 kDa PEG lysostaphin conjugate was more active than the kDa conjugate. The small loss in activity observed with the 2:1 PEG ratios is an acceptable trade-off for the increased serum half-life of this conjugate and its reduced immunogenicity. Potential benefits of these conjugates include, but are not limited to, reduced dosing frequency, reduced ability to induce antibody, retention of activity in subjects with anti-lysostaphin antibodies, and reduced toxicity associated with immunogenic reactions.

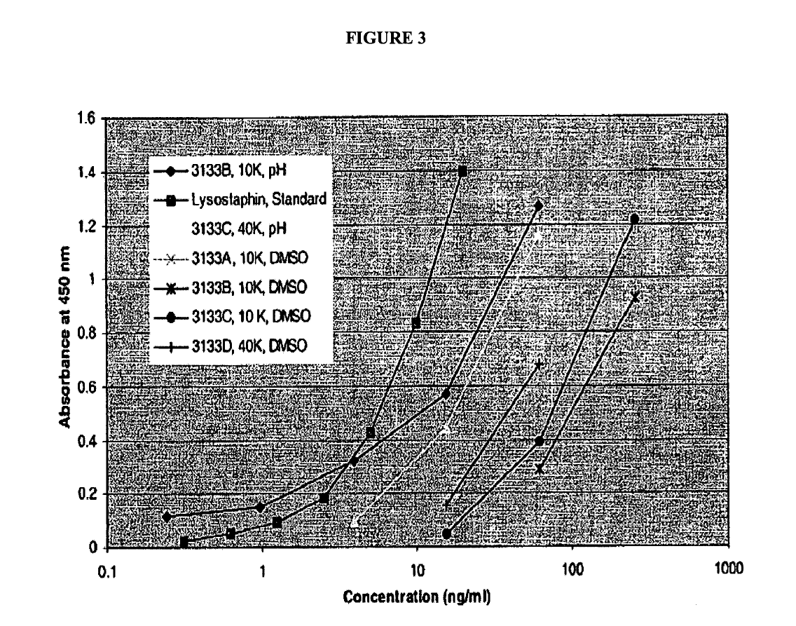

Anti-Lysostaphin Antibody Activity for PEGylated Lysostaphin. The ability of PEG to shield lysostaphin from antibodies was tested in vitro with a lysostaphin-capture immunoassay (See FIG. 3). Unconjugated lysostaphin shows a standard response from 0.3 ng/mL to 20 ng/mL. A heterogeneous response is seen with the binding of PEG-lysostaphin to anti-lysostaphin antibody, but all bind less efficiently than unconjugated lysostaphin. The best shielding observed resulted in a greater than 10-fold reduction in antibody affinity to the PEGylated lysostaphin. This assay environment is different than binding in mucosal surfaces or flowing serum, but it does show that PEG conjugation onto the surface of lysostaphin can at least partially shield the enzyme from antibody binding. There does not appear to be any correlation between enzyme activity and reduced antibody binding, but differences in antibody binding to the 40 kDa and 10 kDa conjugates may be explained by differing degrees of PEGylation. In general, fewer PEG molecules are attached to lysostaphin for the 40 kDa PEG, so it may have a more open structure that does not exclude antibodies as well as the 10 kDa form, which may also explain why the 40 kDa conjugate has better activity. Antibody activity for the 40 kDa conjugate is reduced compared to unconjugated lysostaphin.

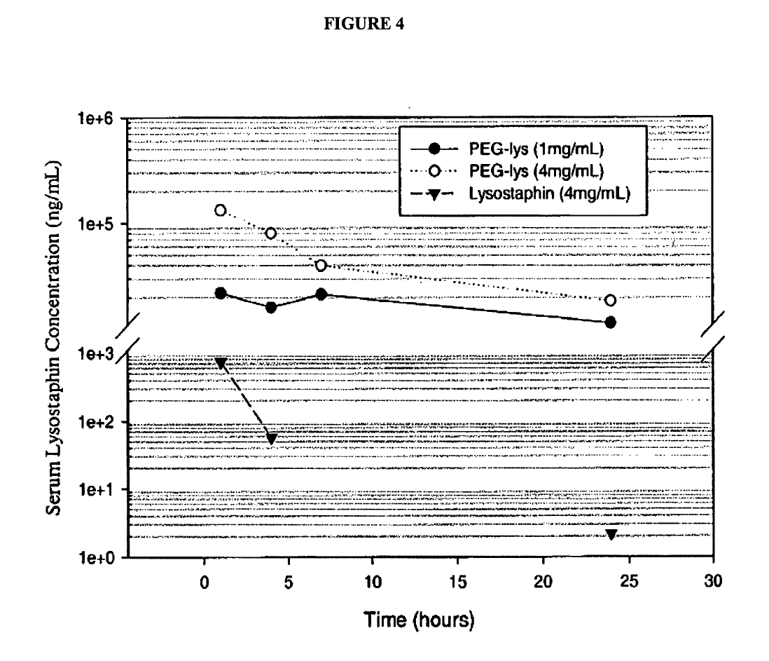

Prolonged Serum Half-Life of PEGylated Lysostaphin. Conjugation of PEG onto protein drugs enables them to avoid the normal clearance mechanisms of the body and thereby leads to increased serum half-life of the drug. The pharmacokinetic profile of lysostaphin with a low degree of PEG modification (1 to 4 PEG's per lysostaphin) was determined in mice and compared to clearance of unconjugated lysostaphin (See FIG. 4). Two enhancements because of PEGylation are apparent from the graph: (1) the half-life of lysostaphin has been dramatically increased and (2) the total serum concentration achieved is much greater than for unconjugated lysostaphin. The serum concentration of the PEG-lysostaphin conjugates drops by only two- to ten-fold over 24 hours whereas unconjugated lysostaphin falls by nearly 500-fold over the same time period. Such a prolonged retention of lysostaphin reduces the dosing frequency needed to remain above therapeutically effective concentrations of the drug.

Maintaining these levels of lysostaphin for longer periods of time also results in more rapid clearance of bacterial infections and decreases the probability that lysostaphin resistance will emerge. Total serum concentrations were also much greater with the PEG-lysostaphin conjugates. At 24 hours post-administration, serum concentrations of PEG-lysostaphin were more than 10 times the concentration of native lysostaphin at just 1 hour post-administration, even when the initial PEG-lysostaphin dose was ¼ that of unconjugated lysostaphin. This result provides that a much lower dose of PEG-lysostaphin may be used to achieve the same or better clinical benefit as conjugated lysostaphin, which results in lower cost of therapy and minimizes potential toxic or allergic reactions to the drug.

### Example 3

### Fractionation of 40 kD PEG Lysostaphin Conjugates

Fractionation of the various 40 kD PEG—lysostaphin conjugate species of Example 2 was performed by ion-exchange chromatography as a means to test enzyme activity as a function of PEG conjugation number. Fractions tended to be enriched in just one specific band. The mono-PEGylated form was purified to greater than 99% 1-mer, while the di-PEGylated form was purified to 93% 2-mer with the remainder contributed mostly by the 1-mer, as determined by size-exclusion chromatography HPLC.

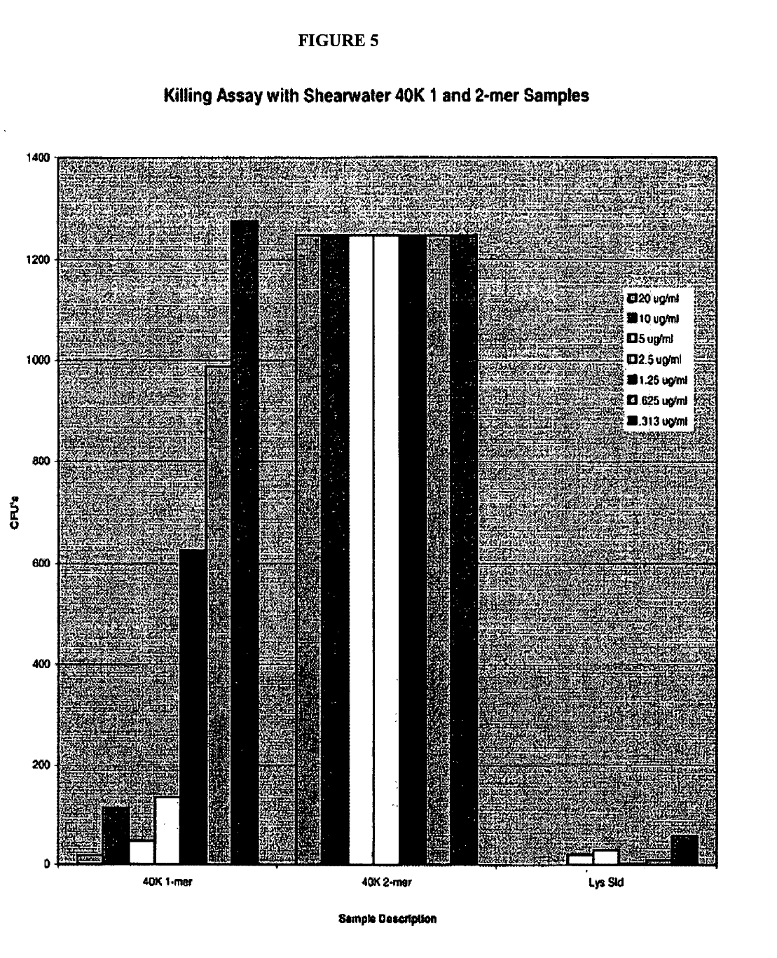

Killing Assay for Activity: The ability of lysostaphin to kill S. aureus (SA) in saline was tested with varying concentrations of the enzyme. The bacteria were streaked onto blood agar plates after a 1-2 hour incubation with lysostaphin and surviving colonies were counted the next day. The data is reported in FIG. 5 as surviving colonies of SA so that the lower value on the graph, the more effective the killing of SA by lysostaphin. The 1-mer has greater activity than the 2-mer, but both have significantly reduced activity compared to unconjugated lysostaphin.

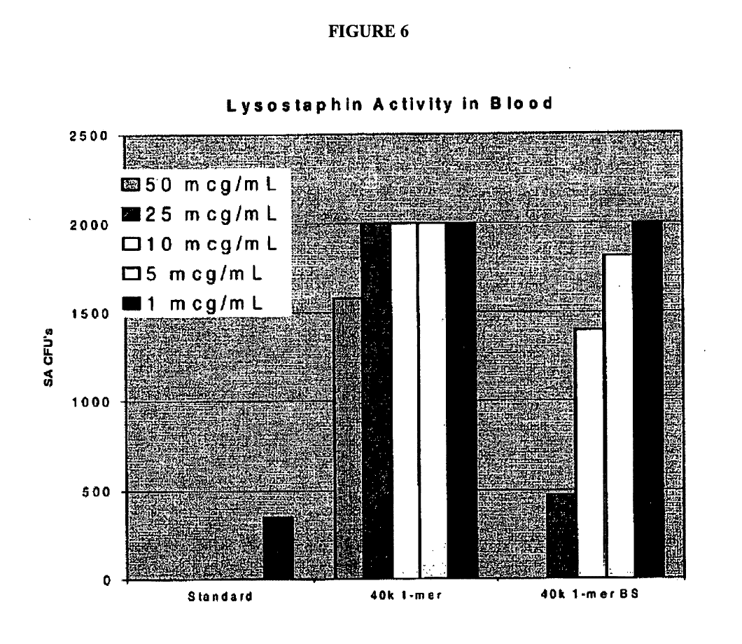

Killing Activity in Blood. The ability of lysostaphin to kill SA in whole, heparinized human blood was tested with varying concentrations of the enzyme. The bacteria were streaked onto blood agar plates after a 1-2 hour incubation with lysostaphin and surviving colonies were counted the next day. The data is reported in FIG. 6 as surviving colonies of SA so that the lower value on the graph, the more effective the killing of SA by lysostaphin. The 40 k 1-mer BS was the Example 1 conjugate made with 50% DMSO. The activity of the 40 k 1-mer is reduced as was observed in the killing assay performed in saline, but the reduction in activity is even greater in blood than in saline.

Several possible explanations for the loss of activity with the 2-mer exist. Although there may be as many as 10 lysine residues available for PEG conjugation onto lysostaphin, each will have a different reactivity, and it is likely that only one or two lysine residues are preferentially PEGylated for a given reaction condition. The preferred site for conjugation of the first PEG chain might lie in a region that is not critical for enzyme function and may explain why there is little loss inactivity for the 1-mer. However, the next-most-preferred lysine for PEGylation may reside in or near the active or cell wall binding sites of lysostaphin, and attachment of PEG to these regions may seriously disrupt enzyme function.

Another possible explanation for the loss of activity with the 2-mer relates to the increased spatial volume of lysostaphin due to PEGylation. Lysostaphin does not act on a soluble, diffusible substrate but rather must be able to penetrate the thick, solid peptidoglycan scaffold of the bacterial cell wall. Each successive addition of PEG raises the molecular weight of lysostaphin, and the increase in spatial volume from the 1-mer to the 2-mer may hinder enzyme access to the pentaglycine cross bridges in the cell wall, thus eliminating its killing activity.

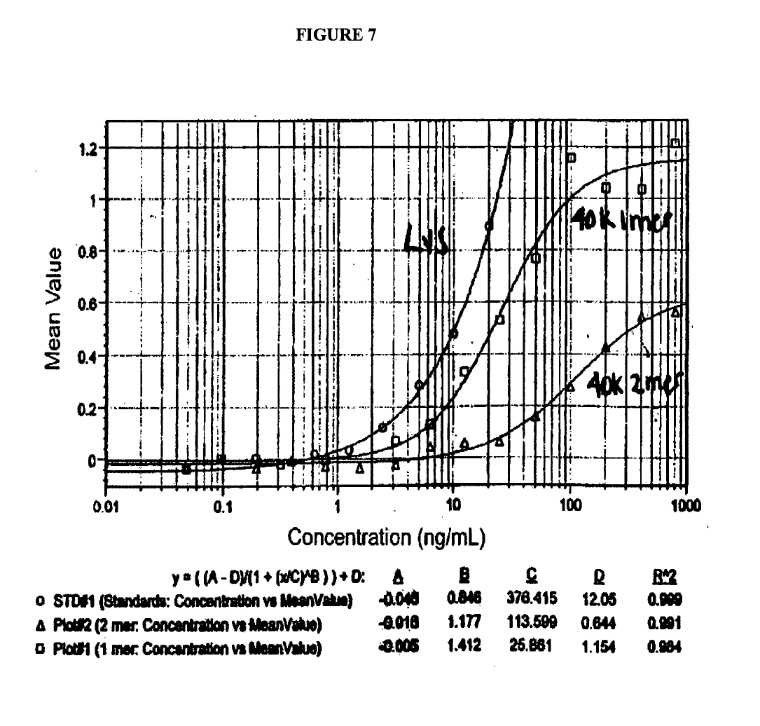

Antibody Reactivity. Reactivity of anti-lysostaphin antibodies to PEGylated lysostaphin was measured by ELISA (See FIG. 7). 96-well plates were coated with a polyclonal anti-lysostaphin antibody (Ab) and then incubated with lysostaphin. Bound lysostaphin was then detected with a polyclonal, HRP-labeled, anti-lysostaphin Ab. The binding level of lysostaphin to these antibodies (Mean Value on y-axis of graph) was determined as a function of enzyme concentration. Both PEG conjugates have reduced Ab binding activity compared to unconjugated lysostaphin, but the 2-mer was much less reactive than the 1-mer.

### Example 4

### Fractionation of 30 kD PEG-Lysostaphin Conjugates

Example 3 was repeated substituting 30 kD PEG for 40 kD PEG and 1-mers and 2-mers of MPEG 30 kD lysostaphin conjugates were isolated in separate fractions.

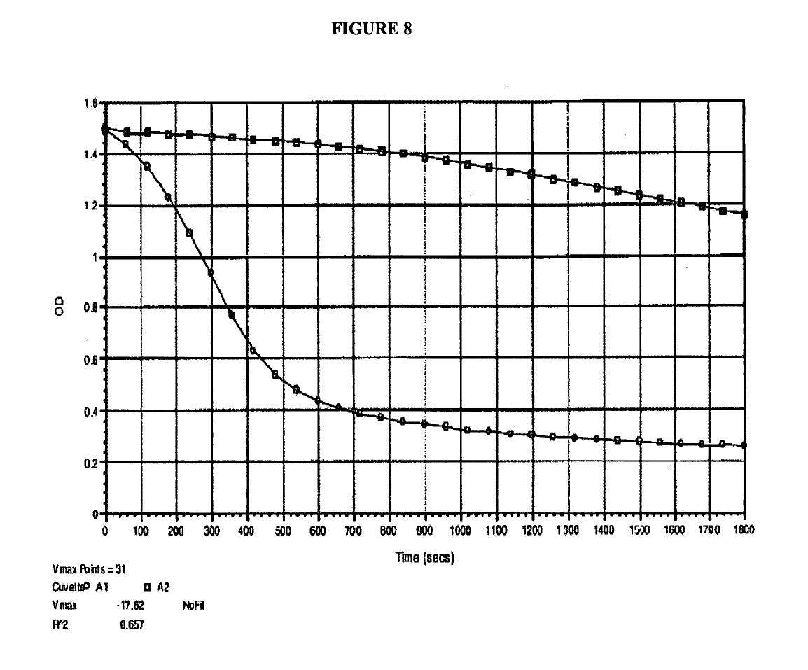

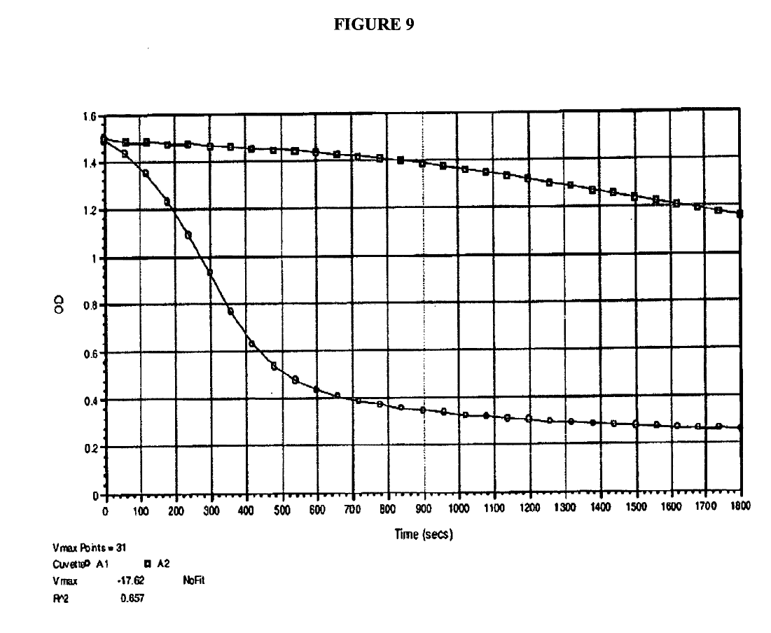

OD drop assay. The OD at 650 nm of a high innoculum of S. aureus (about 10⁹/mL) in saline is monitored over time. When bacteria are lysed, the OD drops and thus is a measure of lysostaphin activity. The faster the OD drops, the greater the enzyme activity. A typical standard takes 6-7 minutes to reach 50% of starting OD. The 1-mer has greater activity than the 2-mer, but both have significantly reduced activity compared to unconjugated lysostaphin (See FIGS. 8 and 9).

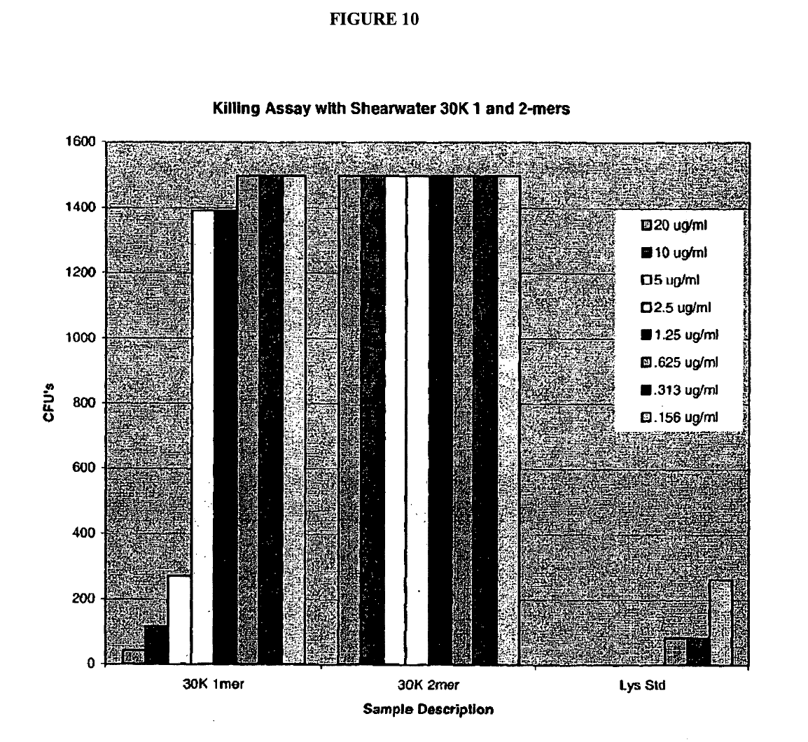

Killing Assay for Activity. The ability of lysostaphin to kill SA in saline was tested with varying concentrations of the enzyme. The bacteria were streaked onto blood agar plates after a 1-2 hour incubation with lysostaphin and surviving colonies were counted the next day. The data is reported as surviving colonies of SA so that the lower value on the graph, the more effective the killing of SA by lysostaphin. The 1-mer has greater activity than the 2-mer, but both have significantly reduced activity compared to unconjugated lysostaphin (See FIG. 10).

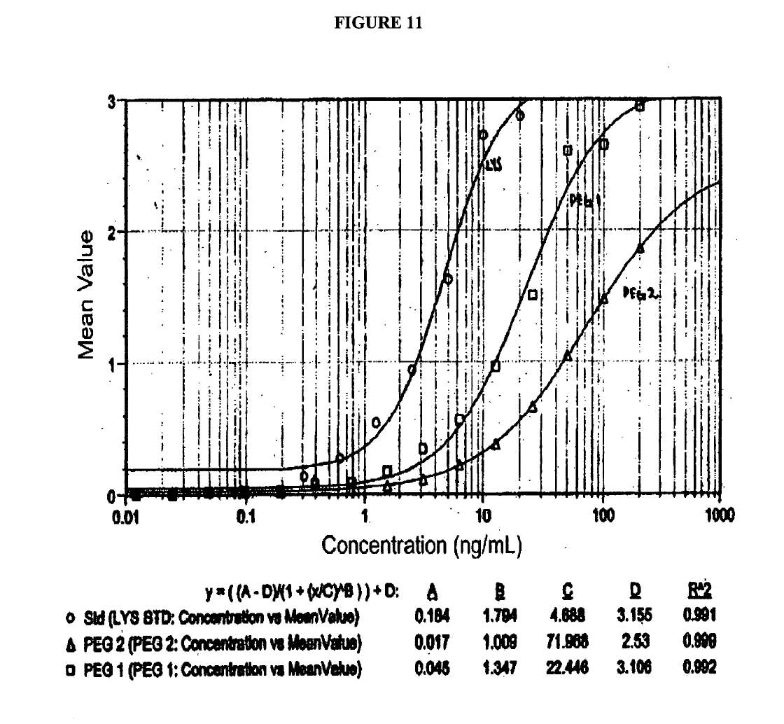

Antibody Reactivity. Reactivity of anti-lysostaphin antibodies to PEGylated lysostaphin was measured by ELISA. 96-well plates were coated with a polyclonal anti-lysostaphin Ab and then incubated with lysostaphin. Bound lysostaphin was then detected with a polyclonal, HRP-labeled anti-lysostaphin Ab. The binding level of lysostaphin to these antibodies (Mean Value on y-axis of graph) was determined as a function of enzyme concentration. The 1-mer has about 7 fold less Ab binding activity and the 2-mer has about 70 fold less Ab binding activity compared to unconjugated lysostaphin (See FIG. 11).

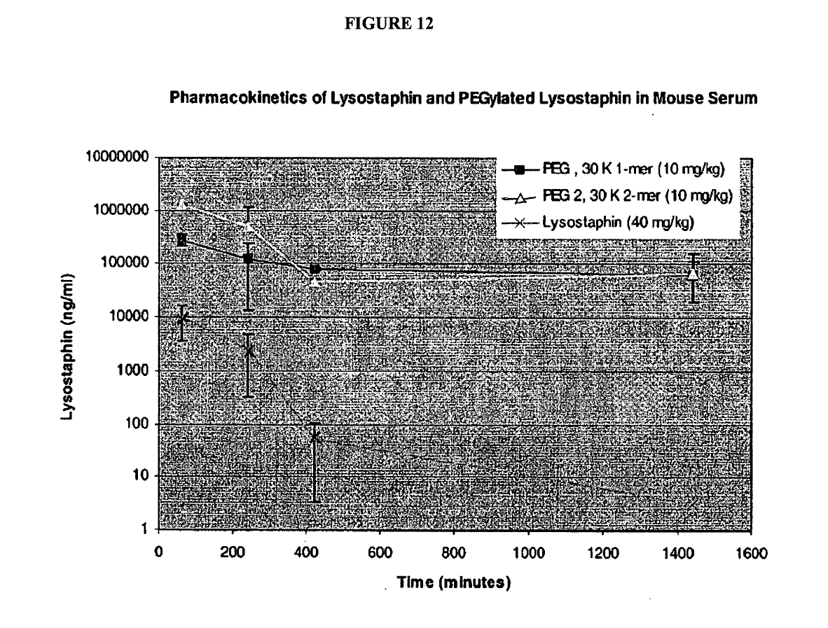

Serum Pharmacokinetics. Mice were injected with standard lysostaphin or PEGylated lysostaphin (30 k 1 and 2-mers) and the serum concentration was determined by ELISA over 24 hours. Higher serum concentrations are achieved with PEGylated enzyme and the half-life of the drug is dramatically increased. The 2-mer achieves higher peak serum lysostaphin concentrations but the long-term persistence seems comparable to that of the 1-mer (See FIG. 12).

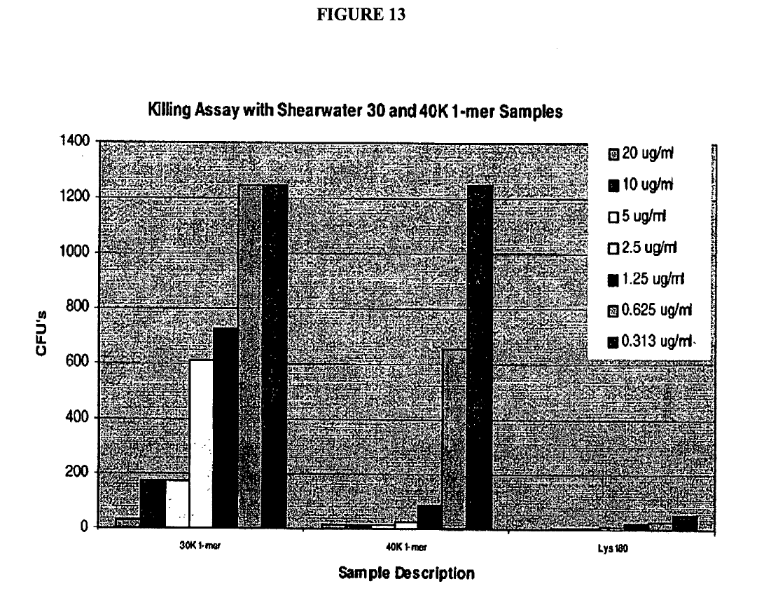

Killing Assay for Activity. The ability of lysostaphin 30 kD and 40 kD PEG 1-mers to kill SA in saline was tested with varying concentrations of the enzyme. The bacteria were streaked onto blood agar plates after a 1-2 hour incubation with lysostaphin and surviving colonies were counted the next day. The data is reported in FIG. 13 as surviving colonies of SA so that the lower value on the graph, the more effective the killing of SA by lysostaphin. The 40 k 1-mer has greater activity than the 30 k 1-mer, but both have significantly reduced activity compared to unconjugated lysostaphin.

### Example 5

### Recombinant Lysostaphin with a Terminal Cys for Polymer Conjugation

A lysostaphin construct, similar to lysostaphin was prepared except that it contained coding for the amino acids Ala-ala-Cys (e.g., similar to “mature” lysostaphin, but containing a terminal cysteine). The procedures described by Goodson et al., Bio Technology, 8, 343 (1990) and Benhar et al., J. Biol. Chem., 269, 13398 (1994) for the insertion of cysteine were followed.

Native lysostaphin does not contain any cysteines. Because the two alanines are un-important to the activity of lysostaphin, this portion of the enzyme could be modified without affecting activity. Thus, in order to conjugate lysostaphin with PEG in a defined and controlled manner, recombinant lysostaphin with a terminal ala-ala-cys was produced in E. coli.

Purification of Cysteine-containing Recombinant Lysostaphin. Cells from a 250 mL culture were harvested by centrifugation and frozen. They were thawed and lysed by extracting the pellet in 70 ml 0.1 M HCl. The extract was centrifuged at 4000 rpm and the supernatant dialyzed overnight at 4° C. against 4 liters PBS diluted 1:2 with water. The dialysate (approximately 150 mL) was further diluted to about 250 mL with water.

Prep 1. Approximately 200 mL of the crude solution was pumped onto a 1 ml SP Sepharose column (PHARMACIA), equilibrated in 12.5 mM sodium phosphate, pH 7. After loading, the column was washed with the equilibration buffer and then eluted with 0.25 M NaCl in 12.5 mM sodium phosphate, pH 7. The eluant was concentrated using an ULTRAFREE 4 (10 kDa cutoff) device, (Millipore) to about 700 μL. Concentration was estimated by adsorption at 280 nm, following 1:20 dilution into PBS, using an extinction coefficient for lysostaphin of 0.49 mg/ml/OD 280:OD 280=0.201×20×0.49 mg/ml/OD 280=2.3 mg/ml. For 27 kDa, this corresponds to 85 μM lysostaphin if 100% pure, for a recovery of 0.7 mL×2.3 mg/mL, or 1.6 mg.

Prep 2. The remaining 50 mL was processed in a similar manner. The concentration of this material was determined as 1.18 mg/mL in a volume of 0.7 L. Recovery=0.82 mg. The total estimated recovery was 1.6±0.8 mg, or 2.4 mg.

Determination of Purity and Presence of Thiol. The free thiol (SH groups) of the recombinant lysostaphin were determined using DTNB (Elhman's reagent) and found to be 23.6 μM. Thus, at least 23.6/85=28% of the lysostaphin contains a free thiol, assuming no other proteins are contributing. SDS PAGE using 8-25% Phast gel (Pharmacia) indicated that a fraction was dimerized, further increasing the percentage of cysteine-lysostaphin obtained upon reduction of the dimer.

Labeling with iodoacetyl biotin, a reagent that reacts only with thiols confirmed that the recombinant lysostaphin contained a cysteine, unlike native lysostaphin. The cysteine can be reacted with reagents such as maleimide-PEG or iodoacetyl-PEG to conjugate lysostaphin at a unique site with PEG.

### Example 6

### NH₂ Terminal Lysostaphin PEGylation by Site-Specific Oxidative Coupling

Lysostaphin has a threonine on its amino terminus. As has been described by Fields et al., Biochem. J., 108, 883 (1968), Gaertner et al., J. Biol. Chem., 269, 7224 (1994), and Geoghegan et al., Bioconj. Chem., 3, 7224 (1992), amino terminus serine or threonine can be oxidized to a glyoxylyl derivative under mild conditions using sodium periodate. This group can then be reacted with amino-oxy PEG, hydrazide PEG or hydrazine PEG to yield lysostaphin pegylated on its amino terminus. An example of this reaction for the pegylation of IL-8 is described in Gaertner et al., Bioconj. Chem., 7, 38 (1996), hereby incorporated by reference.

Amino-oxy PEG (30 kD) is prepared as described in Gaertner et al., Bioconjugate Chemistry or purchased from SHEARWATER. Lysostaphin is prepared at 20 mg/mL in 1% NH₄(HCO₃), pH 8.3 and a 50-fold molar excess of methionine. A 10-fold molar excess of sodium periodate is added. After 10 minutes at room temperature in the dark, the reaction is quenched by the addition of 1/20 volume 50% glycerol. The solution is then dialyzed in the dark against 0.1M sodium acetate, pH 4.6. The solution of oxidized lysostaphin is adjusted to pH 3.6 with 1N acetic acid, then reacted with a 5-fold molar excess of an amino-oxy PEG for 20 hr at room temp in the dark, with gentle stirring. Unreacted PEG is removed by ion exchange chromatography, followed by hydrophobic interaction chromatography to separate unconjugated lysostaphin.

The oxidized lysostaphin may also be functionalized with a reagent such as a (2-thio-pyridyl-cysteine hydrazide (See, e.g., Zara et al, Anal. Biochem., 194, 156 (1991)), which can then be reacted with a thiol reactive PEG such as PEG-maleimide.

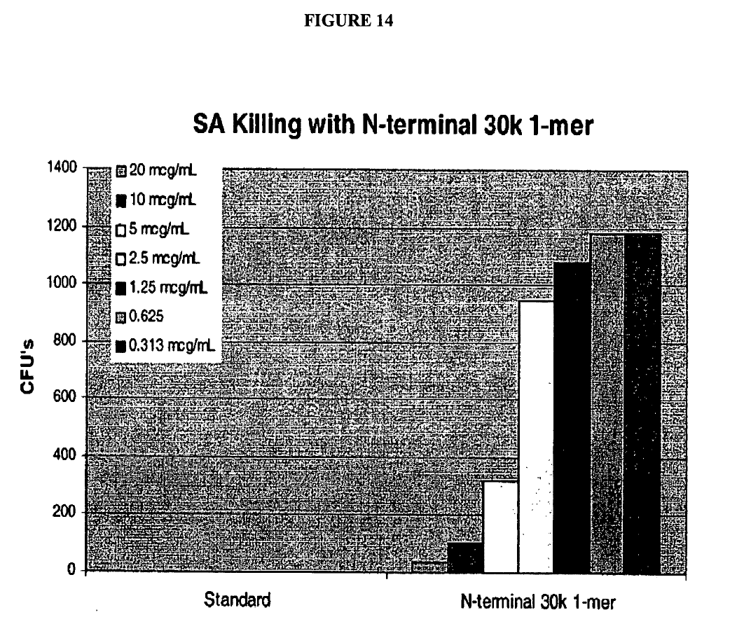

Killing Assay for Activity. The ability of the N-terminal 30 kD PEGlyated lysostaphin to kill SA in saline was tested with varying concentrations of the enzyme. The bacteria were streaked onto blood agar plates after a 1-2 hour incubation with lysostaphin and surviving colonies were counted the next day. The data is reported in FIG. 14 as surviving colonies of SA so that the lower value on the graph, the more effective the killing of SA by lysostaphin. The N-terminal 30 k 1-mer has activity but not at a greater level than previously tested 1-mers (either 30 k or 40 K).

### Example 7

### PEGylation Using Two-Step Heteroligation

Heteroligation chemistry involves labeling component A (in this case lysostaphin) with a reactive group that is capable of reacting only with the reactive group present on component B (in this case, a PEG). In this Example, lysostaphin is chemically modified on a lysine amino groups with a thiol group and then reacted with an electrophilic PEG reagent (e.g., PEG-maleimide), as compared to Example 5, wherein lysostaphin is genetically modified to insert a cysteine group, (which contains a reactive thiol group). Heteroligation chemistry is described in above-referenced Bioconjugate Chemistry.

Lysostaphin was prepared at 20 mg/mL in 75 mM HEPES+2 mM EDTA, pH 7.5 buffer and N-succinimidyl 3-(2-pyridylidithio)propionate (SPDP) (0.1M in DMF) was added dropwise while mixing. The molar ratio of SPDP to lysostaphin was varied in order to vary the degree of labeling. After 1 hr, the pH was reduced by the addition of 1/10 volume 1M sodium acetate, pH 5 and adjusted to pH 5 with 1 N HCl. The solution was made 25 mM by the addition of solid dithiothreitol (DTT). After 15 min., the solution was dialyzed overnight into 10 mM sodium acetate, 2 mM EDTA, pH 5 at 4° C. to remove the DTT. The extent of labeling was determined by the use of DTNB (Ellman's reagent) and from the molar concentration determined by absorbance, at 280 nm using an extinction coefficient of 0.49 mg/ml per absorbance unit at 280 nm.

The thiolated lysostaphin was then reacted with the electrophilic, thiol-selective MPEG-vinylsulfone (SHEARWATER M-VS-5000), mPEG-maleimide (SHEARWATER M-MAL-5000) and mPEG-orthopyridyl disulfide (SHEARWATER M-OPSS-5000). A haloacyl PEG would also be suitable. The reactions were performed at appropriate pH for the PEG reagents to be selective for the thiol. For example, the vinylsulfone addition was performed about pH 7-8. The maleimde addition and disulfide exchange were at performed at pH 6-7.

Excess PEG was removed by ion exchange chromatography. Hydrophobic chromatography could also be used. Lysostaphin containing varying amounts of PEG was thereby fractionated.

The advantages of the two-step method include the ability to limit or control the extent of PEGylation. Additionally, a long chain thiolating reagent can be used (e.g., LC-SPDP, PIERCE, #21651). These reagents allow the thiol group to extend further beyond the protein surface and facilitate conjugation to bulky molecules such as PEG.

A further advantage of the above two-step method is that the thiol will remain reactive for extended periods of time, especially in the absence of oxygen, in EDTA containing buffers and under acidic conditions, all of which minimize oxidation. Likewise, the PEGS described above are all stable at a pH where the reagents are reactive with thiols. This is in contrast to the NHS-ester PEGS that require alkaline conditions to react with amino groups. NHS esters are not stable in base.

Bulky reagents generally react more slowly than small reagents. By employing the two step heteroligation method described in this example, the reaction may be allowed to proceed for an extended period of time and allow for more efficient coupling. This permits less of the PEG reagent to be used, reducing costs. Furthermore, because a higher percentage of the PEG is coupled, purification of the desired PEG-lysostaphin conjugate may be facilitated.

The methods described in the preceding examples can be extended to other anti-microbial proteins, such as nisin. A terminal cysteine can be engineered into a protein as in Example 5, and then coupled to PEG as described in Example 7.

### Example 8

### Characterization of a Cleavable PEG-Lysostaphin Conjugate

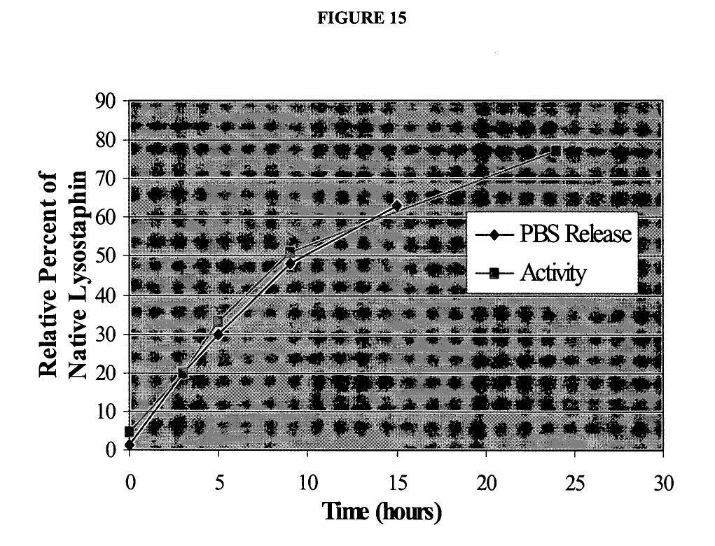

Controlled release of native lysostaphin from a cleavable PEG-Lysostaphin conjugate was analyzed. Cleavage of the PEG-Lysostaphin conjugate was determined by two different methods: measurement of lysostaphin amount by HPLC and measurement of lysostaphin activity by S. aureus killing assay. Results of both assays were reported as the relative percentage of native lysostaphin (conjugate cleaved) to the total lysostaphin in the sample (conjugated plus native) (See FIG. 15). Cleavage occurred during incubation of PEG-lysostaphin in PBS at 37° C. At t=0, nearly all of the lysostaphin is in the conjugated form and has only about 5% relative activity. As native lysostaphin is released from the conjugate, activity increases to match the relative percentage of native lysostaphin in solution, demonstrating that complete lysostaphin activity is restored after PEG is cleaved from the conjugate.

In addition, the ability of an anti-lysostaphin antibody was examined to determine its affinity for binding PEG-conjugated lysostapin as compared to its affinity for binding non-conjugated lysostaphin. An ELISA was performed (e.g., as described in Examples 3 and 4).

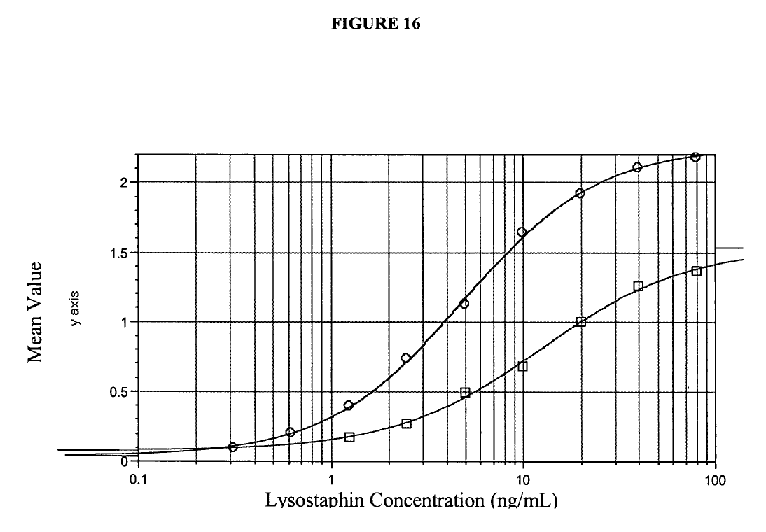

FIG. 16 shows antibody binding to the native versus PEG-conjugated lysostaphin. The circles represent antibody binding to native lysostaphin whereas squares represent antibody binding to PEG-conjugated lysostaphin (wherein ˜100% of the lysostaphin present was conjugated via a degradable attachment to PEG). Thus, an anti-lysostaphin antibody binds significantly less well to PEG-conjugated lysostaphin (e.g., via a degradable attachment) than it does to non-conjugated lysostaphin.

### Example 9

### Serum Pharmacokinetics of a PEG-Lysostaphin Conjugate

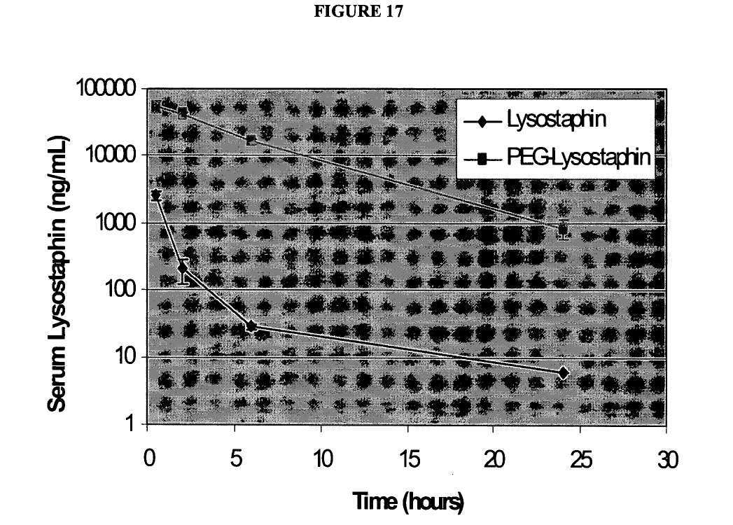

Serum pharmacokinetics of a PEG-lysostaphin were determined (See FIG. 17). Mice were injected with 20 mg/kg of native lysostaphin or a PEG-lysostaphin conjugate. The initial bleed was taken 5 minutes after injection and subsequent bleeds were taken at 1, 6, and 24 hours. The serum lysostaphin concentration was determined by ELISA. The PEG-lysostaphin concentrations represent total lysostaphin content (conjugated plus released native). The initial serum concentrations were more than 10-fold higher for the PEG-lysostaphin. The serum half-life of PEG-lysostaphin was also significantly increased compared to injection of native lysostaphin.

### Example 10

### Efficacy of PEG-Lysostaphin in a Mouse Models of S. Aureus Infection

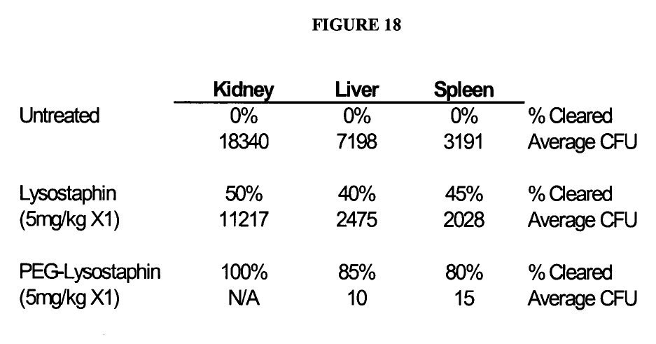

The efficacy (e.g., for treating bacterial infection) of a PEG-lysostaphin conjugate was assessed in two different types of mouse models of infection. In the first, the ability of a PEG-lysostaphin conjugate to treat (e.g. kill and or clear) S. aureus in a mouse model of systemic S. aureus infection was determined. Mice with systemic S. aureus infection were either not treated, or given a single dose (5 mg/kg) of either non-conjugated lysostaphin or a PEG-conjugated lysostaphin. The number of colony forming units (CFUs) of S. aureus present in the kidneys, liver and spleens were then determined. The PEG-lysostaphin conjugate displayed significantly elevated efficacy in clearing S. aureus from the kidney, liver and spleen compared to the efficacy of non-conjugated lysostaphin (See. FIG. 18). Thus, the present invention demonstrates that a single dose of a PEG-lysostaphin conjugate is capable of clearing bacterial infection (e.g., S. aureus infection), and more effectively than unconjugated lysostaphin.

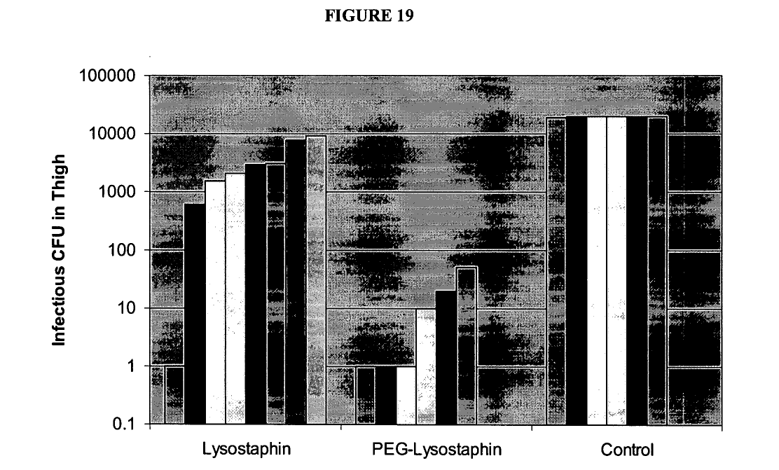

Similarly, the efficacy of a PEG-lysostaphin in a mouse thigh-abscess S. aureus infection was determined. Animals were treated once a day for 3 days with 20 mg/kg of native lysostaphin or PEG-Lysostaphin. After treatment, the number of CFUs present in the thigh tissue was determined. As seen in FIG. 19, the PEG-lysostaphin conjugate was significantly more efficacious than native lysostaphin in clearing (e.g., killing or inhibiting growth of) S. aureus.

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

We claim:

1. A composition comprising polyethylene glycol (PEG) conjugated to lysostaphin or a lysostaphin analogue, wherein the conjugate formed is a degradable conjugate, wherein at least a portion of the antimicrobial activity of said lysostaphin or lysostaphin analogue is retained.

2. The composition of claim 1, wherein said PEG-lysostaphin or PEG-lysostaphin analogue conjugate has a longer in-vivo half-life than non-conjugated lysostaphin or lysostaphin analogue.

3. The composition of claim 1, wherein said PEG-lysostaphin or PEG-lysostaphin analogue conjugate is capable of cleaving cross-linked polyglycine bridges in the cell wall peptidoglycan of Staphylococci.

4. The composition of claim 1, wherein conjugating said lysostaphin or lysostaphin analogue to said polyethylene glycol permits a greater serum concentration of lysostaphin or lysostaphin analogue than is achievable for non-conjugated lysostaphin or lysostaphin analogue.

5. The composition of claim 1, wherein said lysostaphin or lysostaphin analogue is a recombinant lysostaphin or lysostaphin analogue.

6. The composition of claim 5, wherein said recombinant lysostaphin possesses a terminal cysteine.

7. The composition of claim 1, wherein said lysostaphin is naturally derived.

8. The composition of claim 1, wherein said PEG is straight-chained.

9. The composition of claim 1, wherein said PEG is branched.

10. The composition of claim 1, wherein the PEG-lysostaphin conjugate comprises from one to about four polymer molecules per molecule of lysostaphin or lysostaphin analogue.

11. The composition of claim 1, wherein the PEG-lysostaphin conjugate or PEG-lysostaphin analogue conjugate has a mixed degree of conjugation.

12. The composition of claim 1, wherein the PEG-lysostaphin conjugate or PEG-lysostaphin analogue conjugate is a fractionated conjugate.

13. A pharmaceutical composition for treating microbial infection and/or colonization comprising polyethylene glycol (PEG) conjugated to lysostaphin or a lysostaphin analogue, wherein the conjugate formed is a degradable conjugate, wherein at least a portion of the antimicrobial activity of said lysostaphin or lysostaphin analogue is retained, and a pharmaceutically acceptable carrier.

14. The pharmaceutical composition of claim 13, wherein the PEG-lysostaphin or PEG-lysostaphin analogue conjugate is less immunogenic than non-conjugated lysostaphin or lysostaphin analogue.

15. The pharmaceutical composition of claim 13, wherein the PEG-lysostaphin or PEG-lysostaphin analogue conjugate has a greater half-life and/or can attain a greater serum concentration than non-conjugated lysostaphin or lysostaphin analogue.

16. The pharmaceutical composition of claim 13, wherein the PEG-lysostaphin or PEG-lysostaphin analogue conjugate is capable of cleaving the cross-linked polyglycine bridges in the cell wall peptidoglycan of Staphylococci.

17. The pharmaceutical composition of claim 13, further comprising a non-PEG-conjugated antibacterial enzyme.

18. The pharmaceutical composition of claim 17, wherein said non-conjugated antibacterial enzyme is selected from the group consisting of lysostaphin, lysostaphin analogue, lysozyme, mutanolysin, cellozyl muramidase, and combinations thereof.

19. The pharmaceutical composition of claim 13, further comprising an antibiotic.

20. The pharmaceutical composition of claim 19, wherein said antibiotic is selected from the group consisting of β-lactams, cephalosporins, aminoglycosides, sulfonamides, antifolates, macrolides, quinolones, glycopeptides, polypeptides and combinations thereof.

21. A method for the prophylactic or therapeutic treatment of a microbial infection and/or colonization in a subject comprising administering to said subject a pharmaceutical composition comprising polyethylene glycol (PEG) conjugated to lysostaphin or a lysostaphin analogue, wherein the conjugate formed is a degradable conjugate, wherein at least a portion of the antimicrobial activity of said lysostaphin or lysostaphin analogue is retained, and a pharmaceutically acceptable carrier, in an amount effective for preventing or treating said infection and/or colonization.

22. The method of claim 21, wherein said infection is bacterial infection and/or colonization.

23. The method of claim 22, wherein said bacterial infection and/or colonization is caused by bacteria from the genus Staphylococcus.

24. The method of claim 23, wherein said bacteria comprises Staphylococcus aureus.

25. The method of claim 23, wherein said bacteria comprises Staphylococcus epidermidis.
